(19) United States
(12) Patent Application Publication
(10) Pub. No.: US 2026/0209870 A1
(43) Pub. Date: Jul. 23, 2026

(54) VIRCAPSEQ-VERT SYSTEM FOR DIFFERENTIAL DIAGNOSIS, DETECTION, AND SURVEILLANCE OF VIRAL INFECTIONS

(71) Applicant: The Trustees of Columbia University in the City of New York, New York, NY (US)

(72) Inventors: Thomas BRIESE, New York, NY (US); Walter Ian LIPKIN, New York, NY (US)

(21) Appl. No.: 19/569,424

(22) Filed: Mar. 17, 2026

Related U.S. Application Data
(63) Continuation of application No. PCT/US2024/048292, filed on Sep. 25, 2024.
(60) Provisional application No. 63/540,500, filed on Sep. 26, 2023.
(60) Provisional application No. 63/553,273, filed on Feb. 14, 2024.

(51) Int. Cl. C12Q 1/70; C12Q 1/6806; C12Q 1/6813; C12Q 1/6869

(52) U.S. Cl. CPC C12Q 1/702; C12Q 1/6806; C12Q 1/6813; C12Q 1/6869; C12Q 2600/112; C12Q 2600/16

(57) ABSTRACT

A platform for the detection of vertebrate-infecting viruses in a sample, the platform comprising a plurality of oligonucleotide probes, wherein, for each viral taxon included in the platform, the plurality comprises at least one probe partially or fully complementary to a portion of a predetermined coding sequence of said viral taxon, wherein the predetermined coding sequences of the viral taxa, or fragments thereof, cluster at about 60-100% sequence identity, wherein oligonucleotide probes partially or fully complementary to a portion of a predetermined coding sequence within a single viral taxon cluster at about 70-100% sequence identity, wherein each oligonucleotide probe is about 50-300 nucleotides in length, wherein different oligonucleotide probes of the plurality which bind the same predetermined coding sequence are tiled across said predetermined coding sequence at intervals of about 50-500 nucleotides, and wherein the plurality of oligonucleotide probes of the platform comprises 100,000 to 1,500,000 oligonucleotide probes.

### CROSS-REFERENCE TO RELATED APPLICATIONS

This application is a continuation of PCT International Application No. PCT/US2024/048292, filed Sep. 25, 2024, which claims benefit of U.S. Provisional Application No. 63/553,273, filed Feb. 14, 2024, and U.S. Provisional Application No. 63/540,500, filed Sep. 26, 2023, the contents of each of which are hereby incorporated by reference.

### STATEMENT REGARDING FEDERALLY SPONSORED RESEARCH OR DEVELOPMENT

This invention was made with government support under AI109761 awarded by the National Institutes of Health. The government has certain rights in the invention.

Throughout this application, various publications are referenced, including referenced in parenthesis. The disclosures of all publications mentioned in this application in their entireties are hereby incorporated by reference into this application in order to provide additional description of the art to which this invention pertains and of the features in the art which can be employed with this invention.

### BACKGROUND OF THE INVENTION

There is a need in clinical microbiology and public health for assays that enable sensitive and rapid detection of infectious agents. Next Generation Sequencing (NGS) is increasingly used in the fields of oncology and personalized genome medicine but has not gained wider acceptance for clinical microbiology due to operational and bioinformatics complexity, and lower sensitivity compared to agent specific quantitative polymerase chain reaction (qPCR) assays.

### BRIEF SUMMARY OF THE INVENTION

VirCapSeq-VERT is a positive selection system for detection, typing, and strain differentiation of both RNA and DNA viruses with sensitivity comparable to qPCR.

According to embodiments of the present invention, there is provided a virome capture platform for the detection, identification, and/or characterization of vertebrate-infecting viruses in a sample, the platform comprising a plurality of oligonucleotide probes, wherein, for each viral taxon included in the platform, the plurality comprises at least one probe partially or fully complementary to a portion of a predetermined coding sequence of said viral taxon, wherein the predetermined coding sequences of the viral taxa, or fragments thereof, cluster at about 60-100% sequence identity, wherein oligonucleotide probes partially or fully complementary to a portion of a predetermined coding sequence within a single viral taxon cluster at about 70-100% sequence identity, wherein each oligonucleotide probe is about 50-300 nucleotides in length, preferably about 100-300 nucleotides in length, wherein different oligonucleotide probes of the plurality which bind the same predetermined coding sequence are tiled across said predetermined coding sequence at intervals of about 50-500 nucleotides, and wherein the plurality of oligonucleotide probes of the platform comprises 100,000 to 1,500,000 oligonucleotide probes, preferably less than about 1,000,000 oligonucleotide probes.

According to embodiments of the present invention, there is provided a method of screening a sample for vertebrate-infecting viruses, the method comprising:
(a) exposing the sample, or nucleic acids isolated, amplified, and/or enriched from the sample, to any one of the virome capture platforms described herein to form one or more hybridization products, wherein each hybridization product comprises a nucleic acid of the sample and an oligonucleotide probe of the platform;
(b) capturing the one or more hybridization products; and
(c) identifying the presence of one or more virus taxa in the sample based on the sequences of the one or more captured hybridization products;
   thereby screening the sample for vertebrate-infecting viruses.

According to embodiments of the present invention, there is provided a kit comprising any one of the virome capture platforms described herein and instructions for using the platform.

### BRIEF DESCRIPTION OF THE DRAWINGS

FIGS. 1A-1B show a schematic of VirCapSeq-VERT workflow. FIG. 1A. Sample collection, total nucleic acid (TNA) extraction, and library preparation. FIG. 1B. Capture enrichment and sequencing.

FIGS. 2A-2B show linearity data for systemic (FIG. 2A) and respiratory infection (FIG. 2B) test validation. Virus stock was serially diluted in sample matrix (plasma or nasal swabs in VTM, respectively) that had been tested negative for the viruses used for validation, prior to processing for VirCapSeq-VERT.

FIGS. 3A-3B show repeatability and reproducibility for the systemic (FIG. 3A) and respiratory (FIG. 3B) test validation experiments. Negative specimen matrices (plasma or nasal swabs in VTM) were spiked with viruses at 5× their respective LoD (see Table 2). The boxplots represent values for the 10-90 percentile of the normalized viral read counts after host subtraction.

FIGS. 4A-4G display viral genome coverages across a range of viral reads for clinical specimens. FIG. 4A shows CTO22-24 RSV (3.9 million reads, 99.8% coverage). FIG. 4B shows CTO22-37 HRV A (220,000 reads, 99.9% coverage). FIG. 4C shows CTO22-25 ADV C (11,000 reads, 98.4% coverage). FIG. 4D shows CTO22-26 HMPV (6,200 reads, 60% coverage). FIG. 4E shows CTO22-41 RSV (5,592 reads, 59.9% coverage). FIG. 4F shows CTO22-22 HCoV OC43 (1,100 reads, 41% coverage). FIG. 4G shows CTO22-39 ADV C (116 reads, 14% coverage).

FIG. 5 shows a graph displaying the total number of probes selected for viral taxa vs. clustering percentage.

### DETAILED DESCRIPTION OF THE INVENTION

In order to facilitate an understanding of the subject matter disclosed herein, each of the following terms, as used herein, shall have the meaning set forth below, except as expressly provided otherwise herein.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. In embodiments, about means within a standard deviation using measurements generally acceptable in the art. In embodiments, about means a range extending to +/−10% of the specified value. In embodiments, about includes the specified value. Unless otherwise indicated, the word “or” in the specification and claims is considered to be the inclusive “or” rather than the exclusive or, and indicates at least one of and any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a,” “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise,” “include” and “have” and conjugates thereof, are used to indicate that the object or objects of the verb are not necessarily a complete listing of components, elements or parts of the subject or subjects of the verb. Other terms as used herein are meant to be defined by their well-known meanings in the art.

As used herein, the term “viral taxon” shall mean a man-made category for viruses that share certain characteristics, for example, sequence similarity. Many known viral taxa are officially classified by the International Committee on Taxonomy of Viruses (ICTV). Any virus species identified by its partial or complete sequence may be assigned to a viral taxon, e.g., a virus family and/or genus, and included in the platform.

As used herein, the term “vertebrate-infecting virus” shall mean any virus capable of infecting a vertebrate. Such a virus may also infect other organisms, e.g., insects, and/or may be viable in the environment.

As used herein, the term “environmental sample” is a sample obtained from any non-biological media or material(s), including but not limited to, air, soil, water, and swabs of inanimate surfaces. Environmental samples contrast with biological samples, which typically derive from an organism. Examples of biological samples include, but are not limited to, bodily fluids, cells, tissue samples, and swabs of a surface or cavity of a biological organism.

The following embodiments and examples (including details thereof) are set forth to aid in an understanding of the subject matter of this disclosure but are not intended to, and should not be construed to, limit in any way the invention that is claimed.

According to embodiments of the present invention, there is provided a virome capture platform for the detection, identification, and/or characterization of vertebrate-infecting viruses in a sample,
the platform comprising a plurality of oligonucleotide probes, wherein, for each viral taxon included in the platform, the plurality comprises at least one probe partially or fully complementary to a portion of a predetermined coding sequence of said viral taxon,
wherein the predetermined coding sequences of the viral taxa, or fragments thereof, cluster at about 60-100% sequence identity,
wherein oligonucleotide probes partially or fully complementary to a portion of a predetermined coding sequence within a single viral taxon cluster at about 70-100% sequence identity,
wherein each oligonucleotide probe is about 50-300 nucleotides in length, preferably about 100-300 nucleotides in length,
wherein different oligonucleotide probes of the plurality which bind the same predetermined coding sequence are tiled across said predetermined coding sequence at intervals of about 50-500 nucleotides, and
wherein the plurality of oligonucleotide probes of the platform comprises 100,000 to 1,500,000 oligonucleotide probes, preferably less than about 1,000,000 oligonucleotide probes.

In some embodiments, the plurality of oligonucleotide probes of the platform comprises 100,000 to 1,200,000 oligonucleotide probes.

In some embodiments, coding sequences from multiple viral species, strains, or subtypes within a viral taxon are used to generate oligonucleotide probes of the platform. In some embodiments, oligonucleotide probes of the platform which are partially or fully complementary to any region of a viral coding sequence that is classified within a viral taxa included in the platform cluster at about 70-100% identity.

In some embodiments, the virome capture platform is for the detection, identification, and/or characterization of all known or suspected vertebrate-infecting viruses, all known or suspected vertebrate-infecting viral taxa, all known or suspected vertebrate-infecting virus families and/or genera, and/or the known or suspected vertebrate-infecting virome. In some embodiments, the virome capture platform is for the detection, identification, and/or characterization of all known or suspected vertebrate-infecting viruses, all known or suspected vertebrate-infecting viral taxa, all known or suspected vertebrate-infecting virus families and/or genera, and/or the known or suspected vertebrate-infecting virome and comprises 700,000 to 1,000,000 oligonucleotide probes. In some embodiments, the platform for the detection, identification, and/or characterization of viruses that infect particular vertebrate species, e.g. Homo sapiens.

In some embodiments, each oligonucleotide probe is about 100-300 nucleotides in length, preferably 100-150 nucleotides in length.

In some embodiments, each oligonucleotide probe is about 120-300 nucleotides in length.

In some embodiments, the average length of the plurality of oligonucleotide probes is about 125 nucleotides.

In some embodiments, the melting temperature of each oligonucleotide probe is about 50-125° C.

In some embodiments, the melting temperature of each oligonucleotide probe is about 55-115° C.

In some embodiments, the average melting temperature of the plurality of oligonucleotide probes is about 70-100° C., preferably about 85° C.

In some embodiments, different oligonucleotide probes which bind the same predetermined coding sequence are tiled across said predetermined coding sequence at about 60 nucleotide intervals. For example, a set of oligonucleotide probes which each bind different regions of the same coding sequence may be tiled across the predetermined coding sequence at about 60 nucleotide intervals.

In some embodiments, oligonucleotide probes partially or fully complementary to a portion of a hepacivirus coding sequence cluster at about 92% sequence identity.

In some embodiments, oligonucleotide probes partially or fully complementary to a portion of a primate lentivirus coding sequence cluster at about 85% sequence identity.

In some embodiments, the predetermined coding sequences of the viral taxa, or fragments thereof, cluster at about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity.

In some embodiments, each predetermined coding sequence comprises a portion which is partially or fully complementary to an oligonucleotide probe and the portion is about 50-300 nucleotides in length. In some embodiments, each clustered predetermined coding sequence comprises a portion which is partially or fully complementary to an oligonucleotide probe and the portion is about 50-300 nucleotides in length. In some embodiments, each clustered predetermined coding sequence fragment comprises a portion which is partially or fully complementary to an oligonucleotide probe and the portion is about 50-300 nucleotides in length.

In some embodiments, each oligonucleotide probe is at least 80% complementary, preferably at least 90% complementary, to a portion of a predetermined coding sequence.

Where a numerical range is provided herein, it is understood that all numerical subsets of that range, and all the individual integers contained therein, are provided as part of the invention. For example, an oligonucleotide probe which is from 100 to 150 nucleotides in length includes the subset of oligonucleotide probes which are 100 to 140 nucleotides in length, the subset of oligonucleotide probes which are 130 to 150 nucleotides in length etc. as well as an oligonucleotide probe which is 100 nucleotides in length, an oligonucleotide probe which is 101 nucleotides in length, an oligonucleotide probe which is 102 nucleotides in length, etc. up to and including an oligonucleotide probe which is 150 nucleotides in length.

In some embodiments, each viral taxon included in the platform is listed in Table A.

In some embodiments, every viral taxon listed in Table A is included in the platform. Additional viral taxa may be characterized over time based on discovery of new viral sequences and may be included in the platform.

In some embodiments, each viral taxon known or suspected to infect at least one vertebrate, vertebrate organ, or vertebrate organ system is included in the platform.

In some embodiments, the platform is for the simultaneous detection, identification, and/or characterization of all viruses known or suspected to infect at least one vertebrate, vertebrate organ, or vertebrate organ system.

In some embodiments, the virome capture platform is for the detection, identification, and/or characterization of all known or suspected vertebrate-infecting viruses, all known or suspected vertebrate-infecting viral taxa, all known or suspected vertebrate-infecting virus families and/or genera, and/or the known or suspected vertebrate-infecting virome that infect a particular vertebrate organ or vertebrate organ system. For example, the platform may be designed to capture a specific subset of the entire known or suspected vertebrate-infecting virome. In some embodiments, the virome capture platform is for the detection, identification, and/or characterization of all known or suspected vertebrate-infecting viruses, all known or suspected vertebrate-infecting viral taxa, all known or suspected vertebrate-infecting virus families and/or genera, and/or the known or suspected vertebrate-infecting virome that infects a particular vertebrate organ or vertebrate organ system and comprises 100,000 to 500,000 oligonucleotide probes. In some embodiments, the platform is for the detection, identification, and/or characterization of viruses that infect a particular organ or organ system of a vertebrate species, e.g., the respiratory tract of Homo sapiens, or a combination of organ or organ systems of a vertebrate species, e.g., the respiratory tract and gastrointestinal tract of Homo sapiens.

In some embodiments, the viruses infect at least one vertebrate organ or vertebrate organ system.

In some embodiments, the at least one vertebrate organ is selected from the group consisting of skin, liver, brain, lungs, heart, kidney, stomach, intestines, colon, spleen, pancreas, and thyroid.

In some embodiments, the at least one vertebrate organ system is selected from the group consisting of integumentary system, skeletal system, muscular system, nervous system, endocrine system, cardiovascular system, lymphatic system, respiratory system, digestive system, urinary system, and reproductive system.

In some embodiments, the platform is for the simultaneous detection, identification, and/or characterization of viruses that infect at least one vertebrate organ or vertebrate organ system.

In some embodiments, the viruses are selected from the group consisting of respiratory viruses, gastrointestinal viruses, central nervous system (CNS)-infecting viruses, and hepatitis viruses.

In some embodiments, the viruses are selected from the group consisting of parenteral viruses, sexually-transmitted viruses, blood-borne viruses, vector transmitted viruses, oral-fecal viruses, airborne-transmitted viruses, or droplet-transmitted viruses.

In some embodiments, each viral taxon known or suspected to infect at least one vertebrate organ or vertebrate organ system is included in the platform.

In some embodiments, each oligonucleotide probe comprises a capture portion.

In some embodiments, the capture portion is selected from the group consisting of biotin, digoxygenin, a ligand, a small organic molecule, a small inorganic molecule, an aptamer, an antigen, an antibody, and a substrate.

In some embodiments, each oligonucleotide probe is biotinylated.

According to some embodiments of the present invention, there is provided any one of the virome capture platforms described herein and means for capturing, isolating, and/or purifying the plurality of oligonucleotide probes from a mixture of other nucleic acid molecules.

In some embodiments, the oligonucleotides consist of DNA, RNA, bridged nucleic acids, locked nucleic acids, and/or peptide nucleic acids.

In some embodiments, the oligonucleotide probes of the platform may be in solution or attached to a solid support. In some embodiments, the platform comprises oligonucleotide probes generated in an array format, e.g., a cleavable array format. In some embodiments, the platform comprises oligonucleotide probes generated from semiconductor-based synthetic DNA manufacturing.

In some embodiments, the sample is a biological sample or an environmental sample.

In some embodiments, the sample is selected from the group consisting of saliva, mucus, a nasopharyngeal swab, serum, plasma, blood, urine, feces, cerebrospinal fluid, a bodily fluid, cultured cells, an organ tissue, and biopsied tissue.

In some embodiments, the sample is selected from the group consisting of an aqueous sample, a liquid sample, water, wastewater, sewage, greywater, blackwater, freshwater, liquid waste, seawater, drinking water, air, a gaseous sample, soil, a food sample, culture medium, and a swab of an inanimate surface or object.

In some embodiments, the sample is obtained from a sewage system, a drainage system, a plumbing system, or a water treatment facility.

In some embodiments, the sample is obtained from a human subject.

According to embodiments of the present invention, there is provided a method of screening a sample for vertebrate-infecting viruses, the method comprising:
(a) exposing the sample, or nucleic acids isolated, amplified, and/or enriched from the sample, to any one of the virome capture platforms described herein to form one or more hybridization products, wherein each hybridization product comprises a nucleic acid of the sample and an oligonucleotide probe of the platform;
(b) capturing the one or more hybridization products; and
(c) identifying the presence of one or more virus taxa in the sample based on the sequences of the one or more captured hybridization products;
   thereby screening the sample for vertebrate-infecting viruses.

In some embodiments, nucleic acids in the sample are isolated and/or enriched prior to the exposing in step (a).

In some embodiments, the sample is processed prior to the exposing in step (a). For example, the sample may be concentrated or mixed with other reagents.

In some embodiments, the sample is a biological sample or an environmental sample.

In some embodiments, the sample is selected from the group consisting of saliva, mucus, a nasopharyngeal swab, serum, plasma, blood, urine, feces, cerebrospinal fluid, a bodily fluid, cultured cells, an organ tissue, and biopsied tissue.

In some embodiments, the sample is selected from the group consisting of an aqueous sample, a liquid sample, water, wastewater, sewage, greywater, blackwater, freshwater, liquid waste, seawater, drinking water, air, a gaseous sample, soil, a food sample, culture medium, and a swab of an inanimate surface or object.

In some embodiments, the sample is obtained from a sewage system, a drainage system, a plumbing system, or a water treatment facility.

In some embodiments, the sample is obtained from a human subject.

In some embodiments, the method further comprises sequencing one or more detected hybridization products, comparing the nucleotide sequence of the one or more hybridization products to nucleotide sequences of known viruses, and identifying and/or characterizing one or more viruses in the sample based on sequence identity of the hybridization product to the nucleotide sequences of known viruses.

According to embodiments of the present invention, there is provided a kit comprising any one of the virome capture platforms described herein and instructions for using the platform.

In some embodiments, the kit further comprises a sample, wherein the platform is used for the detection, identification, and/or characterization of vertebrate-infecting viruses in the sample.

In some embodiments, the sample is a biological sample or an environmental sample.

In some embodiments, the sample is a liquid sample or an aqueous sample.

In some embodiments, the sample is selected from the group consisting of a water sample, wastewater, sewage, greywater, blackwater, freshwater, liquid waste, seawater, drinking water, air, a gaseous sample, soil, a food sample, culture medium, and a swab of an inanimate surface or object.

In some embodiments, the sample is a wastewater sample or a sewage sample.

In some embodiments, the sample is a wastewater sample.

In some embodiments, the sample is a sewage sample.

In some embodiments, the sample is a liquid sample comprising centralized wastewater.

In some embodiments, the sample is a liquid sample comprising wastewater collected from any source, e.g., wastewater from residences, businesses, industrial sources, or agricultural sources, or any combination of sources. In some embodiments, the sample is a liquid sample comprising wastewater collected from multiple sources.

In some embodiments, the sample is a liquid sample comprising wastewater from a decentralized treatment system.

In some embodiments, the sample is wastewater from a septic tank or septic system.

In some embodiments, the sample is treated with reagents, diluted, or concentrated prior to use with the kit.

In some embodiments, the sample is obtained from a sewage system, a drainage system, a plumbing system, or a water treatment facility.

In some embodiments, the sample is selected from the group consisting of saliva, mucus, a nasopharyngeal swab, serum, plasma, blood, urine, feces, cerebrospinal fluid, a bodily fluid, cultured cells, an organ tissue, and biopsied tissue.

In some embodiments, the sample comprises nucleic acids. In some embodiments, the nucleic acids in the sample are purified, enriched, and/or isolated. The platform of the kit may then be applied to the nucleic acids derived from the sample for the detection, identification, and/or characterization of vertebrate-infecting viruses in the sample.

For the foregoing embodiments, each embodiment disclosed herein is contemplated as being applicable to each of the other disclosed embodiments.

As used herein, all headings are simply for organization and are not intended to limit the disclosure in any manner. The content of any individual section may be equally applicable to all sections. All combinations of the various elements disclosed herein are within the scope of the invention.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only.

### EXAMPLES

### Example 1—Analytical and Clinical Validation of the VirCapSeq-VERT System for Detection of Viruses in Plasma and Nasal Secretions that Cause Systemic and/or Respiratory Infections

### Introduction

A cluster of severe respiratory infections emerged in December 2019, linked to a wet market in Wuhan, China. By the spring of 2020, the world was in the grips of a pandemic with near collapse of health care systems, leading to unprecedented global lockdowns (1, 2). Subsequent reports of Mpox, polio, enterovirus D68 (EV-D68), respiratory syncytial virus (RSV), and measles outbreaks confirmed our vulnerability to the emergence and re-emergence of viral diseases (3-12). Despite the advances made in healthcare and medicine, microbial infections are still one of the leading causes of deaths in both developing as well as developed countries (13). The impact of infectious diseases includes not only morbidity and mortality but a substantive economic burden (14, 15).

Polymerase chain reaction (PCR) has transformed clinical microbiology by providing methods for detection of viruses and quantitation of viral load. It is nonetheless applicable only for detection of known and closely related pathogens and has limited potential for multiplexing. Unbiased Next Generation Sequencing (NGS) is not constrained in multiplex capacity, but has other limitations that include higher cost, longer time to delivery of results, greater complexity of workflow and data analysis, and lower sensitivity (1,000-10,000 copies/ml versus 10-100 copies/ml) (16, 17).

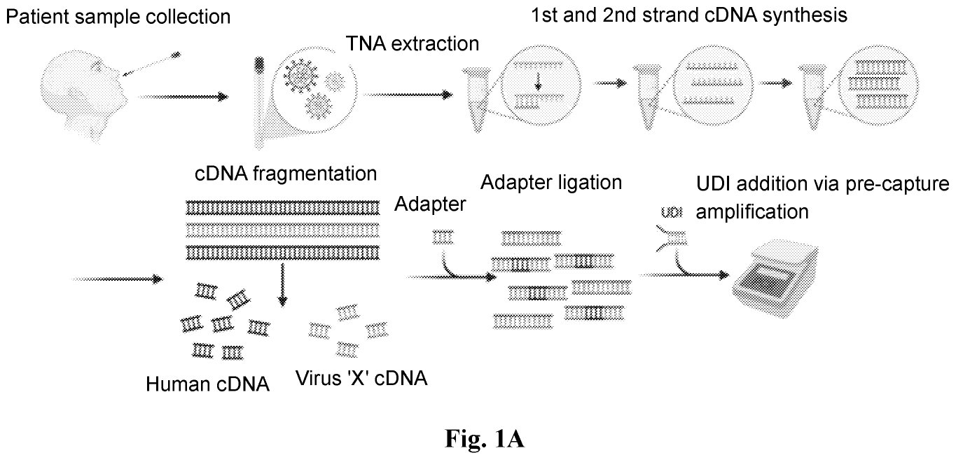

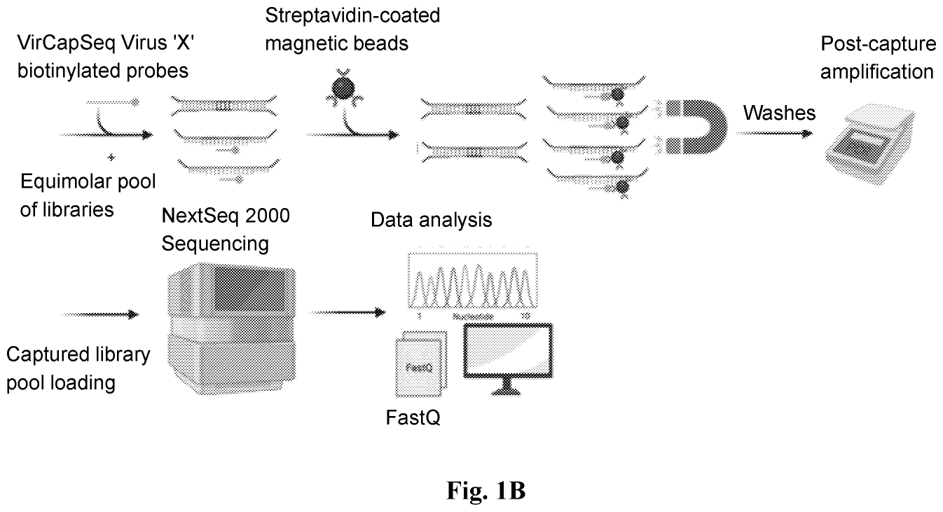

VirCapSeq-VERT is a positive selection system for detection, typing and strain differentiation of both RNA and DNA viruses. It has sensitivity similar to qPCR (5-50 copies/ml), and enables high throughput detailed genomic analyses in less than 36 hours. Total nucleic acid is extracted and subjected to first and second strand cDNA synthesis. Products are sheared prior to library construction. After pooling of bar-coded libraries, viral targets are enriched by VirCapSeq-VERT capture oligonucleotides, followed by washing to remove host products prior to sequencing (schematic in FIGS. 1A-1B). VirCapSeq-VERT has been used with multiple specimen types including saliva, nasopharyngeal swabs, serum, plasma, urine, feces, cerebrospinal fluid, environmental samples, and organ tissues (16, 18-31). The enabling assay component is a library of oligonucleotide probes designed to bind and capture sequences of all known vertebrate viruses. The capture probes cover all relevant sequences in Genbank, RefSeq, EMBL, or GISAID (a listing of viral targets included in VirCapSeq-VERT capture library construction can be found in Table S1 of reference 16; the capture library was updated in May 2021 by adding probes for newly reported sequence entries, including the novel SARS-CoV 2 virus). With the goal of extending this method to clinical microbiology a rigorous assessment of the performance characteristics of VirCapSeq-VERT was undertook, including its limit of detection (LoD), repeatability and reproducibility, differential diagnosis in mixed infections, clinical accuracy and precision. This assessment was undertaken to obtain formal approval for use of VirCapSeq-VERT as a certified diagnostic test by the New York State Department of Health (NYSDOH) under the auspices of the Clinical Laboratory Evaluation Program (CLEP), a stringent state licensure program run by the NYSDOH (32).

### Materials and Methods

### Viruses

A representative set of viruses with RNA and DNA genomes that vary in polarity, size, and structure, and are relevant to the practice of clinical microbiology were selected for validation (Table 1).

Validation was performed using a mix of contrived samples, controls, and clinical specimens. Virus culture supernatants were procured either pre-quantified or were quantified in copies per milliliter using Conformitè Europëenne (CE) marked commercial qPCR kits from Siemens Healthineers (Resp21 FTD Plus and Neuro9 FTD) with the exceptions of severe acute respiratory syndrome coronavirus 2 (SARS-CoV-2) and Zika virus (ZIKV) which were quantified using FDA Emergency Use Authorization (EUA) approved assays (33, 34). To replicate conditions found in clinical materials, live virus was spiked into appropriate negative matrices that were confirmed to be negative for the viruses included in the validation. The only exception being SARS-CoV-2 where for safety reasons pre-extracted total nucleic acid (TNA) from both the virus and pooled nasal swabs were mixed. Viruses or TNA were spiked into the background at desired concentrations for all performance characteristics studies.

### Controls

Negative controls: Negative controls included either single donor plasma samples or a pool of anterior nasal swabs in viral transport media (VTM) (Becton Dickinson, USA; Copan, Italy) that were confirmed in specific PCR assays to be free of nucleic acids of viruses used in validation studies.

Positive control: The External RNA Controls Consortium (ERCC, ThermoFisher Scientific, USA) has created a commercial kit for use as a common set of RNA controls for various molecular platforms including NGS (35). ERCC spike-in mix 1 was used in a salmon sperm DNA background as a technical post-extraction control to monitor performance of the library and capture hybridization workflow as well as spillover/contamination events of sample sequences (viral or human) into ERCC/salmon sperm or vice versa. The capture probe set was designed to include probes matching only half of the ERCC RNA control set that represented the various template sizes and concentrations included in the set to specifically measure efficacy of the capture enrichment.

### Extractions

TNA was extracted either from 300 μl of plasma or 250 μl of anterior nasal swabs collected in VTM on the NucliSENS easyMAG platform (bioMérieux, France). A no-template control (nuclease-free water) was included in each extraction run.

### Library Preparation, Capture Hybridization, and Sequencing

For the plasma samples, libraries were prepared as per our published protocol (16). For analyses of respiratory samples, Twist library preparation kits and Twist Fast Hybridization Reagents (Twist Biosciences, USA) were employed. Amplified and purified barcoded libraries were pooled and set up for the capture using VirCapSeq-VERT probes and using either Roche Hybridization Kits (Roche, USA) or Twist Fast Hybridization Reagents (Twist Biosciences, USA). The enriched pools were sequenced on Illumina NextSeq instruments. Our target range was 10 million raw reads per sample.

### Creating Contrived Samples and Analytical Validation Studies Designs

Analytical validation was done with contrived samples, comprised of quantified live cultured viral isolates or viral TNA from SARS-CoV-2, spiked into negative background matrices. Single donor plasma from five individuals sourced from the New York Blood Center and nasal swabs collected from 10 anonymized volunteers in 2 ml VTM were screened by VirCapSeq-VERT. A sample was defined as negative if it did not contain sequences of any of the viruses used in the validation. The acceptable negative background matrices used were one single donor plasma and a pool of nasal swabs in VTM. The viral isolates were sourced from our own archived repository and from commercial sources (ATCC, USA; Zeptometrix, USA). Viruses were quantified and serially diluted in 10-fold dilutions, ranging from 500,000 copies/ml to 0.5 copies/ml for nasal swabs and from 500,000 copies/ml to 5 copies/ml for plasma, due to the limited single donor sample volume.

Using the LoD data, repeatability and reproducibility, and accuracy in differential diagnosis of mixed infections were determined. Repeatability and reproducibility assays were done at 5×LoD for each virus. Following guidance from CLEP, three independent runs, with each run containing three replicates of all viruses, were conducted by three different operators, starting from distinct extractions, on three separate starting dates. For mixed infections, samples were generated that contained one agent at LoD and another at higher concentration.

### Clinical Concordance

Diagnostic concordance between VirCapSeq-VERT and approved qPCR assays were examined. Clinical samples (plasma and nasal swabs in VTM) were sourced through the New York Presbyterian Hospital (NYP) network and from Associated Regional and University Pathologists, Inc. (ARUP Labs, USA). ARUP samples included 30 plasma specimens that had been tested using qPCR for the following viruses: BK Polyomavirus (BKPyV), human herpesvirus 5 (HHV-5), hepatitis B virus (HBV), human parvovirus B19 (B19V), human herpesvirus 4 (HHV4), human herpes virus 6 (HHV6), and hepatitis C virus (HCV) based on availability. Additionally, to test assay performance for viruses for which no relevant clinical specimens were available, live virus at concentrations ranging from 10,000-100 copies/ml were seeded in the negative plasma diluent to create contrived specimens (ten adenovirus C (ADV-C), five coxsackievirus B4 (CV-B4), six enterovirus A71 (EV-A71), ten human herpesvirus 1 (HHV-1), eleven ZIKV, two BKPyV, ten (HBV), and four B19V). For respiratory infections, 50 banked and de-identified clinical nasal swab specimens that had been previously tested for a range of respiratory viruses were tested.

### Data Analysis, Interpretation, and Reporting

Sequencing data sets were analyzed using an automated bioinformatics pipeline and custom viral database. Demultiplexed fastq files had adapters trimmed using Cutadapt program (36). The reads were then quality filtered and end-trimmed with PRINSEQ software (37). The host reads were removed by mapping quality-filtered reads against a custom host reference database using Bowtie2 mapper (38). Then reads that match any of these criteria were removed: shorter than 50 bases after adapter trimming; reads with Q scores below 25; low complexity reads with an entropy value ≤70; and those with 20 or more Ns. The first 9 bases of the remaining reads were trimmed as the quality tends to be inherently poor. The final host-subtracted reads were subjected to homology search using GenBank MegaBLAST against a curated custom viral database that was created by downloading viral sequences from GenBank, and removing faulty, and misannotated sequences. All reads were subsequently re-mapped to the reference viral sequences identified by the initial homology search to validate the MegaBLAST results. Finally, stringency filters including e-value of ≤1.00E-35, identity of ≥95%, alignment lengths of ≥100 nucleotides (nt), minimum number of unique reads aligned, minimum number of regions ≥100 nt, and cumulative minimum length of ≥500 nt were applied. All viral sequences that were identified at this stage were included in the final results report.

### Results

### Limits of Detection and Linearity

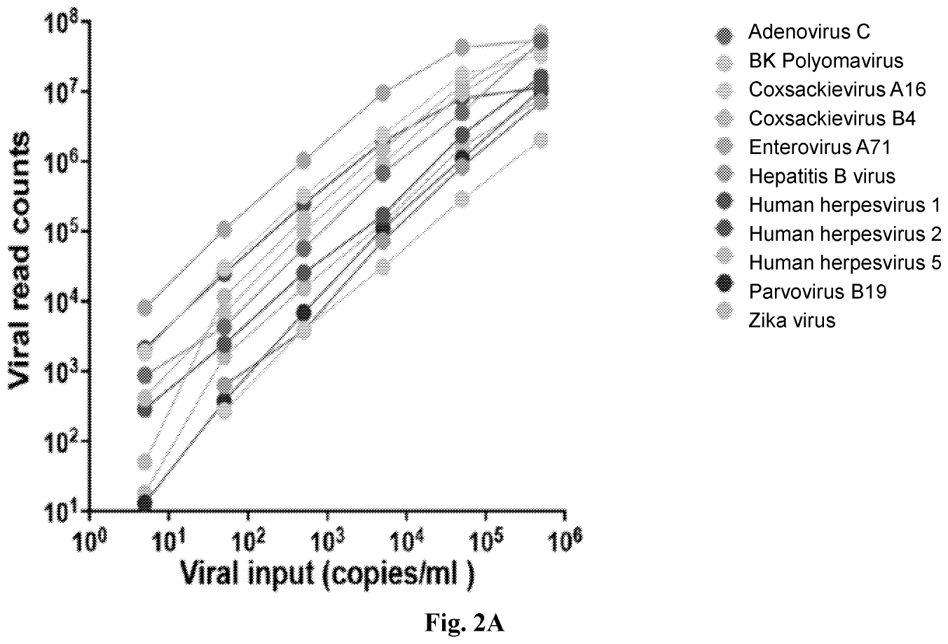

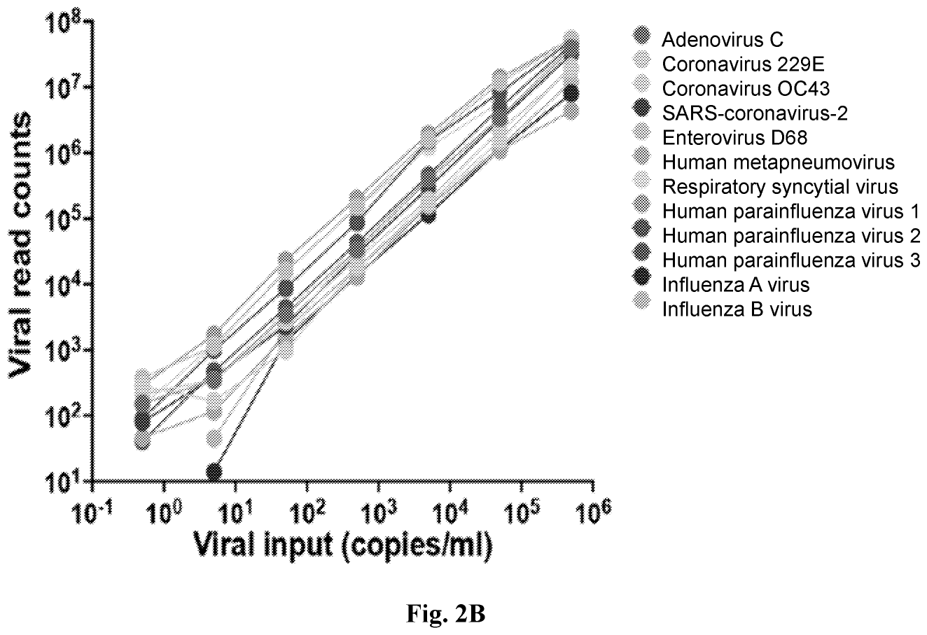

LoDs and performance of VirCapSeq in the context of virus quantity were determined using qPCR quantitated virus stocks or TNA (SARS-CoV-2) serially 10-fold diluted in the respective background matrix (plasma or nasal swab in VTM), starting at a concentration of 500,000 copies/ml. A LoD of 5 copies/ml was determined in plasma for ADV-C, coxsackieviruses (CV) A16 and B4, EV-A71 and HHV 1 and 5, and in nasal swabs for EV-D68 and human parainfluenza viruses (HPIV) 1 and 2. A LoD of 50 copies/ml was determined in plasma for BKPyV, HBV, HHV-2, B19V, ZIKV, and in nasal swab for ADV-C, coronaviruses 229E, OC43 and SARS-CoV-2, human metapneumovirus (HMPV), RSV, HPIV-3, and influenza viruses (FLUV) A and B. Linearity data for plasma and respiratory swab samples are shown in FIGS. 2A-2B and LoDs are listed in Table 2. Tables S1 and S2 show raw and normalized read counts, and the fraction of viral reads per sample.

### Repeatability and Reproducibility

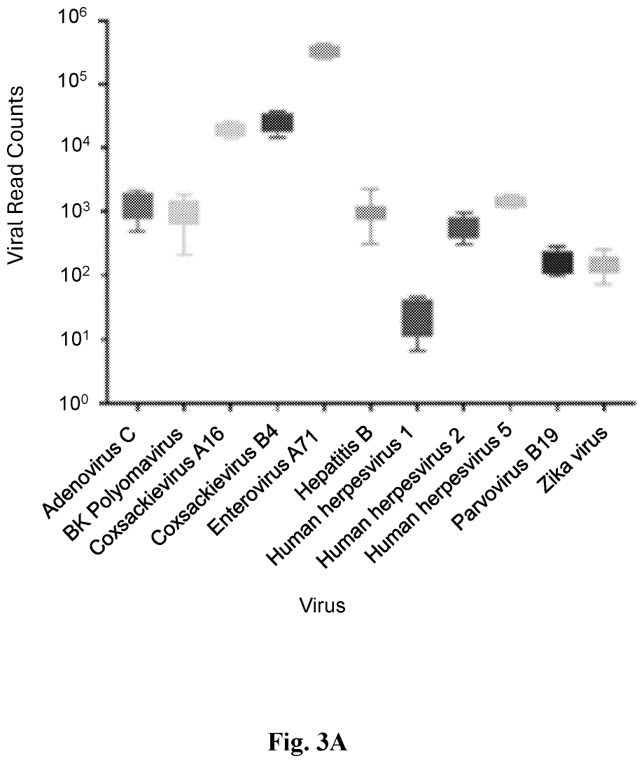

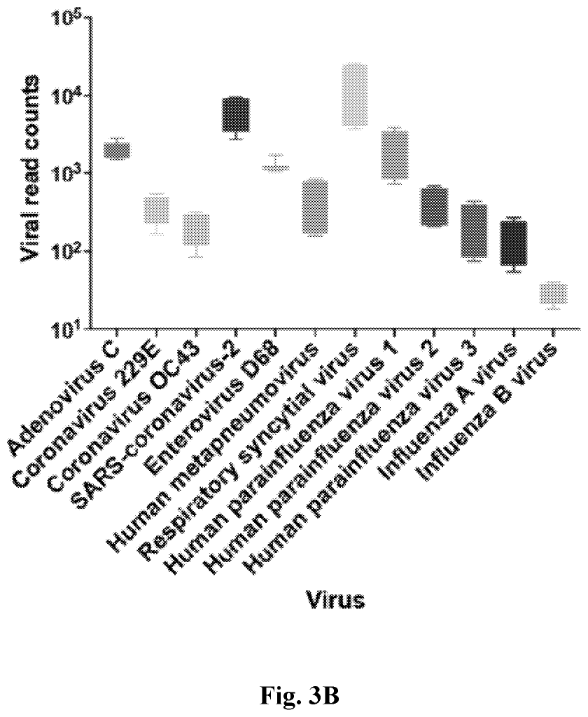

Based on the LoD information, contrived specimens were created at 5×LoD for each virus for measuring intra-run repeatability and inter-run reproducibility. Three independent runs, each containing triple replicates of every virus, were conducted by three different operators, starting from independent extractions, on three separate starting dates. VirCapSeq-VERT achieved a high degree of precision for all viruses tested with an overall coefficient of variance of 18% for plasma and 10% for respiratory swab samples. Data for both sample types are plotted in FIGS. 3A-3B.

### Diagnostic Concordance

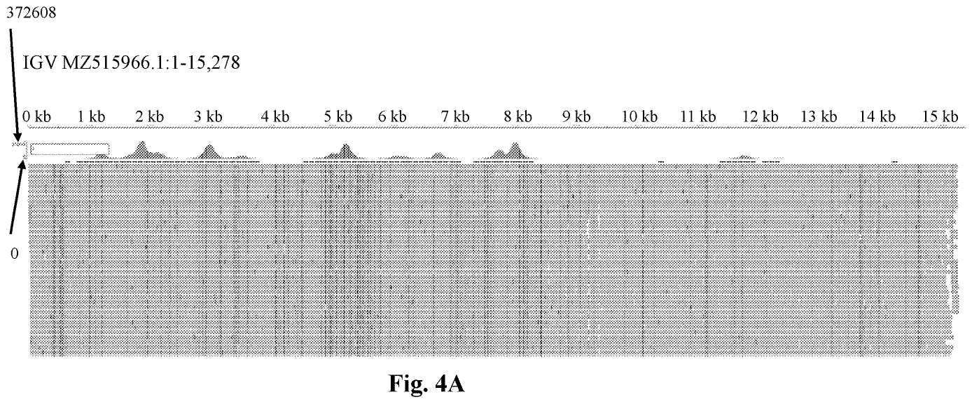

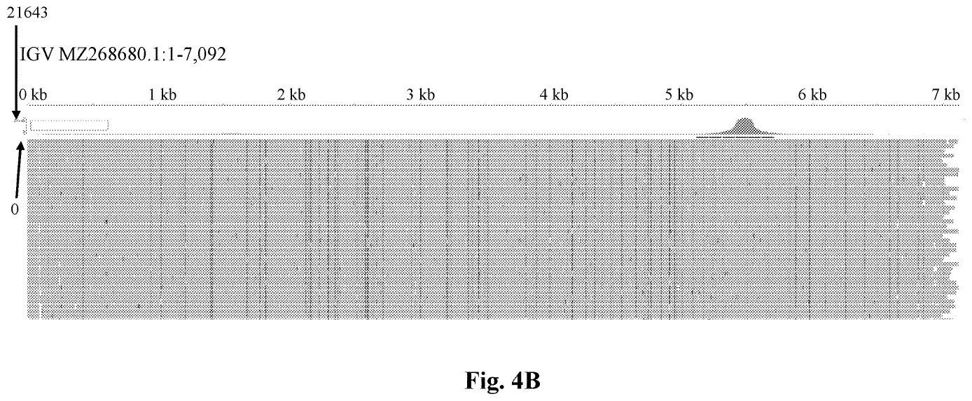

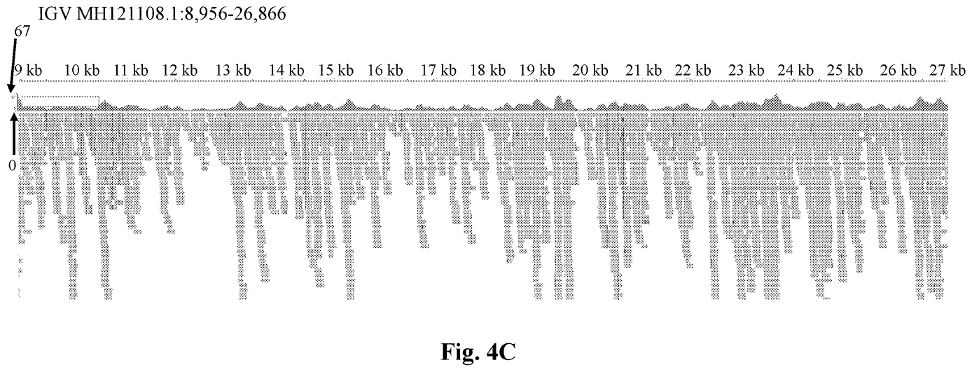

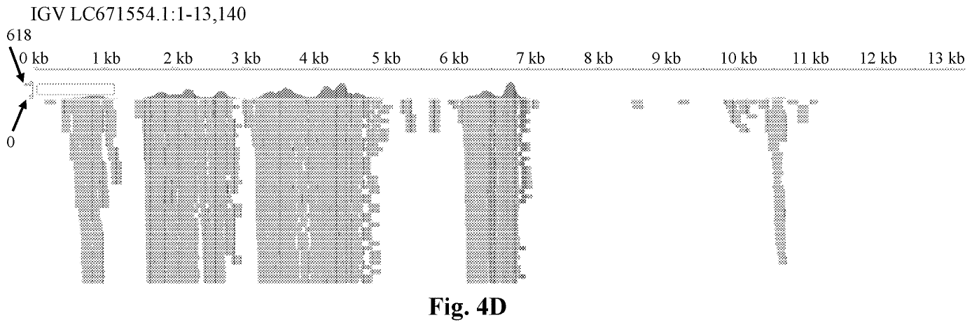

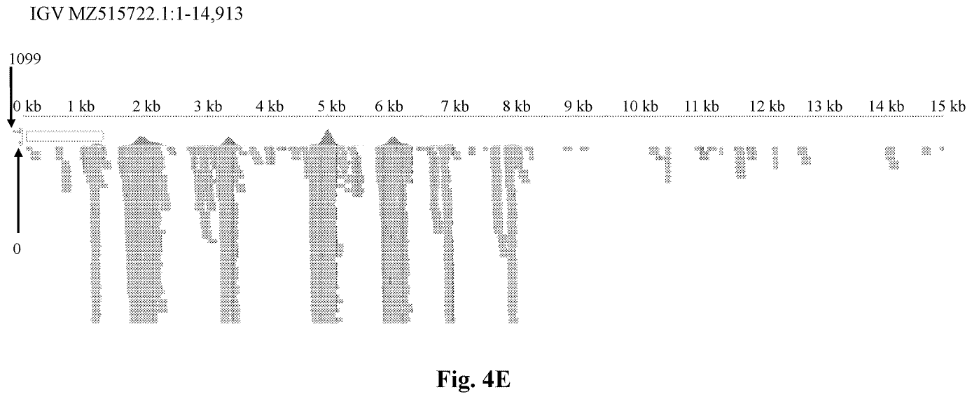

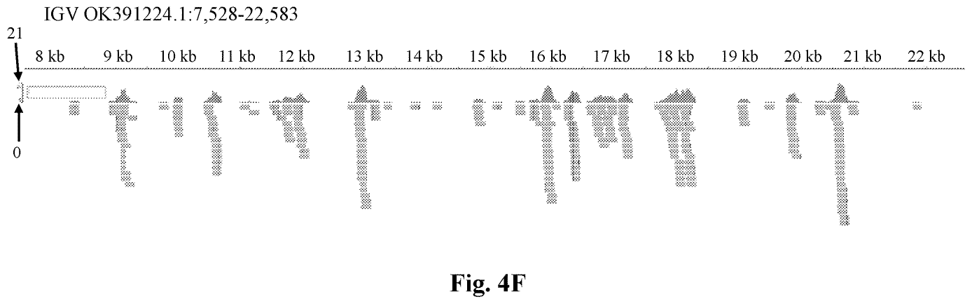

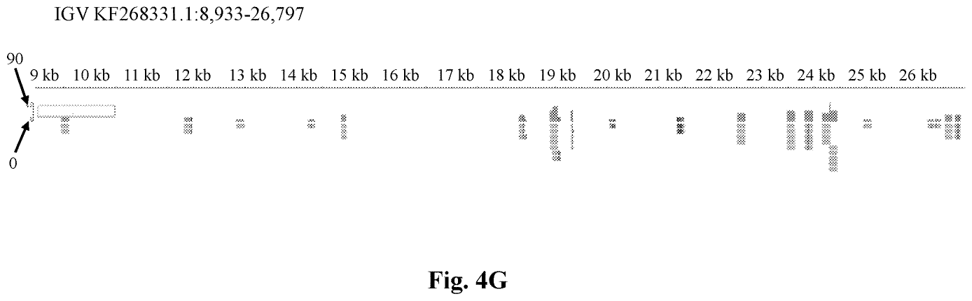

For systemic infections clinical specimens sourced commercially were tested. Clinical specimens positive for additional viruses to those used for analytical validation (Table 1) were included based on availability. These were HHV-4, HHV-6, and HCV positive specimens. Findings with these specimens provided additional support for the utility of the platform in detecting and differentiating between related members within a virus family, subfamily, or genus. VirCapSeq-VERT results matched previous diagnoses with the exception three HHV-4 samples and two HBV samples. The provided qPCR Ct values for the negative HHV-4 samples were 42.7, 42.1 and 40.6. The negative HBV samples had a load <10 and 20 IU. Independent qPCR with CE-cleared tests were conducted, which confirmed the VirCapSeq-VERT results, and it was concluded that discordance may reflect sample degradation from the date of the initial clinical testing and receipt for VirCapSeq-VERT analysis. CLEP advised for creation of additional contrived material for viruses not adequately represented in the available clinical samples. Table 4 lists data for all plasma samples examined during clinical validation. VirCapSeq-VERT demonstrated a 100% clinical sensitivity and specificity on the valid samples. For respiratory infections, 50 banked clinical nasal swab specimens that were diagnosed for respiratory pathogens were tested using the BioFire Diagnostics FilmArray Respiratory Panel. Sample processing and data analysis were blinded but results of the initial molecular characterization were provided. Some of these samples had co-infections at the time of initial testing (see Table S3). There were instances where the purported virus was not detected by VirCapSeq-VERT analysis. Accordingly, additional qPCR tests were conducted to examine whether discordance represented failure of VirCapSeq-VERT. Ten of eleven discordant samples were found to be negative in subsequent CE or FDA EUA qPCR assays. These results suggest that discordance was due to sample degradation between the initial tests and later VirCapSeq-VERT and qPCR analyses. One of the discordant samples was positive for adenovirus sequence in qPCR after 36 cycles, demonstrating a low viral load in the specimen. Tables 5 and S3 summarize results of the clinical specimen testing and concordance between the BioFire assay and VirCapSeq-VERT, and results of CE or FDA EUA confirmation qPCR assays for negative discordant specimens. Viral genome coverages across a range of viral reads for a few clinical specimens are displayed in FIGS. 4A-4G.

VirCapSeq-VERT provided insights that were not achieved with PCR assays and in two instances detected co-infections (RSV in one sample and influenza C in another, Table S3) that were not indicated by the initial tests. For nasal swab testing VirCapSeq-VERT demonstrated significant concordance for valid specimens with a clinical sensitivity of 98% and specificity of 100%.

### Discussion

NGS has transformed the field of pathogen discovery and surveillance (39-42). It has nonetheless not been widely applied in clinical microbiology laboratories due to cost, complexity, insensitivity, and lengthy turnaround times. Capture sequencing addresses each of these limitations. Through positive selection, relevant targets are enriched, while non-relevant nucleic acids from the host and environment are substantially reduced. This approach results in two to three orders of magnitude enhancement in sensitivity at a reduced cost. An additional advantage of capture sequencing is that it mitigates Health Insurance Portability and Accountability Act (HIPAA) concerns because host sequence data are not collected. In previous studies, VirCapSeq-VERT has enabled differential diagnosis of unexplained febrile illnesses in Tanzania, clusters of severe respiratory infections in Uganda, as well as meningitis in the UK, and myocarditis in Canada and Switzerland (19, 23, 25, 29). These studies were conducted under the research use only designation because at the time the work was conducted, NGS based infectious diseases assays, including VirCapSeq-VERT, were not certified as diagnostic tests. A critical step in the transition of NGS from research facilities to clinical microbiology laboratories is validation by regulatory agencies based on demonstration of assay performance. The validation of the VirCapSeq-VERT platform for detecting viral nucleic acid in plasma and nasal swabs is reported here. With LoDs established at ≤50 copies/ml it has sensitivity comparable to qPCR, is highly reproducible, can detect multiple viruses in a single sample, and matches the results obtained with accepted gold standard assays such qPCRs and FilmArrays.

A key challenge encountered in the validation efforts was accurate quantitation of the viral cultures. Other groups have reported sensitivity in terms of plaque forming units or 50% tissue culture infectious dose (43-46). Both of these methods are cumbersome and subjective, and at times yield conflicting results for identical starting cultures in a direct comparison (47, 48). Other methods used for virus quantitation include qPCR and western blots (47). Though faster and easier to use, qPCRs nevertheless pose a similar challenge where two independent assays targeting the same virus can yield differing titers (46, 49). This discrepancy was observed firsthand when multiple PCR assays were used in parallel. For consistency, titers quantified by regulatory approved assays were used. One limitation of the validation is that the resources to conduct additional 2-fold dilutions or Probit analyses to determine LoDs with better accuracy were not available and instead established them based on current data for the 10-fold dilutions.

VirCapSeq-VERT achieved results that are highly concordant with other molecular platforms currently used in clinical microbiology laboratories. No information about the nature of the PCR assays that were used for the initial tests on plasma samples procured from ARUP Labs was available. The respiratory specimens were previously tested using BioFire FilmArrays. It has been reported that the BioFire FilmArray is unable to differentiate between adenovirus species and between entero- and rhinoviruses (43). As anticipated, VirCapSeq-VERT detected as well as enabled species identification for these viruses in clinical specimens. Additionally, VirCapSeq-VERT identified co-infections that were not indicated in the clinical tests.

Clinical samples also included sequences of anelloviruses; herpesviruses HHV-6, -7, -8; papillomaviruses, and endogenous retroviruses. Their significance is uncertain as they are not known to be associated with the acute febrile illness that led to clinical analysis.

VirCapSeq-VERT is designed to detect all vertebrate viruses. It is not feasible to test all known viruses; however, the representative viruses tested varied in genome structure and length, and included both RNA and DNA viruses. The strength of this platform is that it is not limited to pathogens with DNA genomes (50), a limited repertoire of pathogens (51), and that it has sensitivity comparable to qPCR and has obtained regulatory approval for clinical testing from the NYSDOH. Additionally, although regulatory approval for expedited processing has not yet been obtained, preliminary data to indicate that the time required from sample receipt to agent identification can be reduced from the current 24-36 hours to around 12 hours is available. As the NGS field continues to evolve it is anticipated that clinicians will have access to actionable data in a time frame that reduces mortality, morbidity, and health care costs.

TABLE 1
Genome structures and sizes for 22 representative viruses
used for validation study in plasma and nasal swab samples.

| Plasma | | | Nasal swab | | |
|---|---|---|---|---|---|
| | Genome | Genome | | Genome | Genome |
| Virus | Type | Size (nt) | Virus | Type | Size (nt) |
| Adenovirus 1 | dsDNA | 40000 | Adenovirus 1 | dsDNA | 40000 |
| | linear | | | linear | |
| BK Polyomavirus | dsDNA | 5000 | Coronavirus 229E | ss(+) RNA | 30000 |
| | linear | | | | |
| Coxsackievirus A16 | ss(+) RNA | 7400 | Coronavirus OC43 | ss(+) RNA | 30000 |
| Coxsackievirus B4 | ss(+) RNA | 7300 | SARS-coronavirus-2 | ss(+) RNA | 30000 |
| Enterovirus A71 | ss(+) RNA | 7000 | Enterovirus D68 | ss(+) RNA | 7000 |
| Hepatitis B | dsDNA | 3200 | Human | ss(−) RNA | 14000 |
| | circular | | metapneumovirus | | |
| Human | dsDNA | 152000 | Respiratory | ss(−) RNA | 15000 |
| herpesvirus 1 | linear | | syncytial virus | | |
| Human | dsDNA | 155000 | Human parainfluenza | ss(−) RNA | 16000 |
| herpesvirus 2 | linear | | virus 1 | | |
| Human | dsDNA | 240000 | Human parainfluenza | ss(−) RNA | 16000 |
| herpesvirus 5 | linear | | virus 2 | | |
| Parvovirus B19 | ssDNA | 5600 | Human parainfluenza | ss(−) RNA | 16000 |
| | | | virus 3 | | |
| Zika virus | ss(+) RNA | 11000 | Influenza A virus | ss(−) RNA | 13500 |
| | | | Influenza B virus | ss(−) RNA | 14500 |

TABLE 2
Limits of Detection (LoD) for 22 representative viruses used
for validation study in plasma and nasal swab samples.

| Plasma | | Nasal swab | |
|---|---|---|---|
| | LoD | | LoD |
| Virus | (copies/ml) | Virus | (copies/ml) |
| Adenovirus 1 | 5 | Adenovirus 1 | 50 |
| BK Polyomavirus | 50 | Coronavirus 229E | 50 |
| Coxsackievirus A16 | 5 | Coronavirus OC43 | 50 |
| Coxsackievirus B4 | 5 | SARS-Coronavirus-2 | 50 |
| Enterovirus A71 | 5 | Enterovirus D68 | 5 |
| Hepatitis B | 50 | Human | 50 |
| | | metapneumovirus | |
| Human | 5 | Respiratory | 50 |
| herpesvirus 1 | | syncytial virus | |
| Human | 50 | Human parainfluenza | 5 |
| herpesvirus 2 | | virus 1 | |
| Human | 5 | Human parainfluenza | 5 |
| herpesvirus 5 | | virus 2 | |
| Parvovirus B19 | 50 | Human parainfluenza | 50 |
| | | virus 3 | |
| Zika virus | 50 | Influenza A virus | 50 |
| | | Influenza B virus | 50 |

TABLE 3
Detection of co-infections in nasal swab samples.
Co-infections

| | | | Concentration | | | | |
|---|---|---|---|---|---|---|---|
| Mix | Virus | Viral Load | Copies/ml | Detected | Total reads | Viral Reads | Viral Reads/M |
| M1 | Influenza A virus | LoD | 5.00E+01 | + | 41,026,700 | 1,050 | 26 |
| | Coronavirus OC43 | High | 5.00E+04 | + | | 35,158,111 | 856,957 |
| M2 | Influenza A virus | High | 5.00E+04 | + | 3,517,029 | 110,729 | 31,484 |
| | Coronavirus OC43 | LoD | 5.00E+01 | + | | 1,524 | 434 |
| M3 | Adenovirus C | LoD | 5.00E+01 | + | 4,486,231 | 17,335 | 3,864 |
| | Human metapneumovirus | High | 5.00E+04 | + | | 1,112,083 | 247,888 |
| M4 | Adenovirus C | High | 5.00E+04 | + | 7,830,480 | 3,934,006 | 502,397 |
| | Human metapneumovirus | LoD | 5.00E+01 | + | | 1,253 | 161 |
| M5 | Human parainfluenza virus 2 | LoD | 5.00E+00 | + | 18,636,157 | 1,117 | 60 |
| | Respiratory syncytial virus | High | 5.00E+03 | + | | 12,985,557 | 696,794 |
| M6 | Human parainfluenza virus 2 | High | 5.00E+03 | + | 4,563,732 | 1,327,348 | 290,847 |
| | Respiratory syncytial virus | LoD | 5.00E+00 | + | | 105,840 | 23,192 |
| M7 | Influenza B virus | LoD | 5.00E+01 | + | 2,994,401 | 493 | 165 |
| | Human parainfluenza virus 3 | High | 5.00E+04 | + | | 123,490 | 41,240 |
| M8 | Influenza B virus | High | 5.00E+04 | + | 2,698,984 | 122,712 | 45,466 |
| | Human parainfluenza virus 3 | LoD | 5.00E+01 | + | | 170 | 63 |
| M9 | Coronavirus 229E | LoD | 5.00E+01 | + | 22,887,286 | 4,578 | 200 |
| | Enterovirus D68 | High | 5.00E+04 | + | | 18,213,661 | 795,799 |
| M10 | Coronavirus 229E | High | 5.00E+03 | + | 5,676,372 | 2,873,451 | 506,213 |
| | Enterovirus D68 | LoD | 5.00E+00 | + | | 5,660 | 997 |
| M11 | Human parainfluenza virus 1 | LoD | 5.00E+00 | + | 2,687,212 | 2,394 | 891 |
| | SARS-coronavirus-2 | High | 5.00E+03 | + | | 2,530,082 | 941,527 |
| M12 | Human parainfluenza virus 1 | High | 5.00E+04 | + | 21,726,230 | 7,880,254 | 362,707 |
| | SARS-coronavirus-2 | LoD | 5.00E+01 | + | | 31,929 | 1,470 |
| M13 | Influenza A virus | High | 5.00E+04 | + | 25,261,588 | 72,567 | 2,873 |
| | Respiratory syncytial virus | High | 5.00E+04 | + | | 6,748,813 | 267,157 |
| | Influenza B virus | High | 5.00E+04 | + | | 143,347 | 5,675 |
| | SARS-coronavirus-2 | High | 5.00E+04 | + | | 8,634,842 | 341,817 |

TABLE 4
Data demonstrating diagnostic concordance with qPCR assay for plasma specimens.

| | # of specimen tested | VirCapSeq | In-house qPCR results |
|---|---|---|---|
| | (true clinical + | results | on discordant samples |
| Virus | contrived) | (positive/tested) | (positive/tested) |
| Adenovirus 1 | 10 (0 + 10) | 10/10 | |
| BK polyomavirus | 10 (8 + 2) | 10/10 | |
| Coxsackievirus B4 | 5 (0 + 5) | 5/5 | |
| Enterovirus A71 | 6 (0 + 6) | 6/6 | |
| Human herpesvirus 4* | 4 (4 + 0) | 1/4 | 0/3 |
| Human herpesvirus 5 | 4 (4 + 0) | 4/4 | |
| Human herpesvirus 1 | 10 (0 + 10) | 10/10 | |
| Human herpesvirus 6* | 4 (4 + 0) | 4/4 | |
| Hepatitis B virus | 14 (4 + 10) | 12/14 | 0/2 |
| Hepatitis C virus* | 2 (2 + 0) | 2/2 | |
| Human parvovirus B19 | 8 (4 + 4) | 8/8 | |
| Zika virus | 11 (0 + 11) | 11/11 | |

*Included based on clinical specimen availability but not part of the validation test.

TABLE 5
Data demonstrating diagnostic concordance with BioFire
FilmArrays for the respiratory specimens.

| | BioFire | VirCapSeq | In-house qPCR results |
|---|---|---|---|
| | results | results | on discordant samples |
| Virus in specimen | (positive/tested) | (positive/tested) | (positive/tested) |
| Adenovirus | 12/12 | 9/11 | 1/3 |
| Coronavirus OC43 | 3/3 | 3/3 | |
| SARS-coronavirus-2 | 1/1 | 0/1 | 0/1 |
| Enterovirus/rhinovirus | 14/14 | 13/14 | 0/1 |
| Influenza A virus | 5/5 | 4/5 | 0/1 |
| Human metapneumovirus | 4/4 | 4/4 | |
| Human parainfluenza virus 3 | 5/5 | 4/5 | 0/1 |
| Respiratory syncytial virus | 15/15 | 12/13 | 0/3 |

TABLE S1
Linearity data for plasma tests.

| | Raw | Viral | Normalized | % viral |
|---|---|---|---|---|
| Sample Name | reads | reads | viral reads/M | reads |
| Adenovirus C | 2473571 | 866 | 350 | 0.04 |
| 5.00E+00 cps/ml | | | | |
| Adenovirus C | 2326169 | 4,316 | 1855 | 0.19 |
| 5.00E+01 cps/ml | | | | |
| Adenovirus C | 2596315 | 56,761 | 21862 | 2.19 |
| 5.00E+02 cps/ml | | | | |
| Adenovirus C | 3350481 | 680,425 | 203083 | 20.31 |
| 5.00E+03 cps/ml | | | | |
| Adenovirus C | 7863696 | 4,982,548 | 633614 | 63.36 |
| 5.00E+04 cps/ml | | | | |
| Adenovirus C | 63485608 | 52,343,730 | 824498 | 82.45 |
| 5.00E+05 cps/ml | | | | |
| BK-Polyomavirus | 4243796 | 0 | 0 | 0.00 |
| 5.00E+00 cps/ml | | | | |
| BK-Polyomavirus | 4538610 | 268 | 59 | 0.01 |
| 5.00E+01 cps/ml | | | | |
| BK-Polyomavirus | 5063301 | 3,936 | 777 | 0.08 |
| 5.00E+02 cps/ml | | | | |
| BK-Polyomavirus | 4397841 | 30,550 | 6947 | 0.69 |
| 5.00E+03 cps/ml | | | | |
| BK-Polyomavirus | 5475822 | 286,423 | 52307 | 5.23 |
| 5.00E+04 cps/ml | | | | |
| BK-Polyomavirus | 7804037 | 2,050,977 | 262810 | 26.28 |
| 5.00E+05 cps/ml | | | | |
| Coxsackievirus A16 | 2369915 | 1,839 | 776 | 0.08 |
| 5.00E+00 cps/ml | | | | |
| Coxsackievirus A16 | 2298935 | 29,700 | 12919 | 1.29 |
| 5.00E+01 cps/ml | | | | |
| Coxsackievirus A16 | 2923152 | 326,793 | 111795 | 11.18 |
| 5.00E+02 cps/ml | | | | |
| Coxsackievirus A16 | 4900529 | 2,404,785 | 490719 | 49.07 |
| 5.00E+03 cps/ml | | | | |
| Coxsackievirus A16 | 24512584 | 17,612,027 | 718489 | 71.85 |
| 5.00E+04 cps/ml | | | | |
| Coxsackievirus A16 | 43518573 | 32,831,406 | 754423 | 75.44 |
| 5.00E+05 cps/ml | | | | |
| Coxsackievirus B4 | 2166496 | 50 | 23 | 0.00 |
| 5.00E+00 cps/ml | | | | |
| Coxsackievirus B4 | 2134868 | 11,510 | 5391 | 0.54 |
| 5.00E+01 cps/ml | | | | |
| Coxsackievirus B4 | 2479875 | 152,122 | 61343 | 6.13 |
| 5.00E+02 cps/ml | | | | |
| Coxsackievirus B4 | 4190148 | 1,479,260 | 353033 | 35.30 |
| 5.00E+03 cps/ml | | | | |
| Coxsackievirus B4 | 17991512 | 12,168,630 | 676354 | 67.64 |
| 5.00E+04 cps/ml | | | | |
| Coxsackievirus B4 | 92037490 | 67,349,837 | 731765 | 73.18 |
| 5.00E+05 cps/ml | | | | |
| Enterovirus A71 | 3746646 | 8,012 | 2138 | 0.21 |
| 5.00E+00 cps/ml | | | | |
| Enterovirus A71 | 5399507 | 106,688 | 19759 | 1.98 |
| 5.00E+01 cps/ml | | | | |
| Enterovirus A71 | 5761147 | 1,031,454 | 179036 | 17.90 |
| 5.00E+02 cps/ml | | | | |
| Enterovirus A71 | 16926627 | 9,450,290 | 558309 | 55.83 |
| 5.00E+03 cps/ml | | | | |
| Enterovirus A71 | 56363790 | 42,877,489 | 760728 | 76.07 |
| 5.00E+04 cps/ml | | | | |
| Enterovirus A71 | 70974878 | 54,153,018 | 762989 | 76.30 |
| 5.00E+05 cps/ml | | | | |
| Hepatitis B virus | 4973002 | 0 | 0 | 0.00 |
| 5.00E+00 cps/ml | | | | |
| Hepatitis B virus | 5560374 | 624 | 112 | 0.01 |
| 5.00E+01 cps/ml | | | | |
| Hepatitis B virus | 6239690 | 3,784 | 606 | 0.06 |
| 5.00E+02 cps/ml | | | | |
| Hepatitis B virus | 3687408 | 72,768 | 19734 | 1.97 |
| 5.00E+03 cps/ml | | | | |
| Hepatitis B virus | 6427163 | 840,659 | 130798 | 13.08 |
| 5.00E+04 cps/ml | | | | |
| Hepatitis B virus | 15337652 | 7,037,029 | 458807 | 45.88 |
| 5.00E+05 cps/ml | | | | |
| Human herpesvirus | 4509090 | 2,099 | 466 | 0.05 |
| 1 5.00E+00 cps/ml | | | | |
| Human herpesvirus | 4833526 | 24,855 | 5142 | 0.51 |
| 15.00E+01 cps/ml | | | | |
| Human herpesvirus | 4829849 | 245,406 | 50810 | 5.08 |
| 1 5.00E+02 cps/ml | | | | |
| Human herpesvirus | 7596240 | 1,903,558 | 250592 | 25.06 |
| 1 5.00E+03 cps/ml | | | | |
| Human herpesvirus | 23358088 | 7,973,058 | 341340 | 34.13 |
| 1 5.00E+04 cps/ml | | | | |
| Human herpesvirus | 37101921 | 11,380,098 | 306725 | 30.67 |
| 1 5.00E+05 cps/ml | | | | |
| Human herpesvirus | 4926983 | 289 | 59 | 0.01 |
| 2 5.00E+00 cps/ml | | | | |
| Human herpesvirus | 6078255 | 2,426 | 399 | 0.04 |
| 2 5.00E+01 cps/ml | | | | |
| Human herpesvirus | 5510642 | 25,222 | 4577 | 0.46 |
| 2 5.00E+02 cps/ml | | | | |
| Human herpesvirus | 4103992 | 168,234 | 40993 | 4.10 |
| 2 5.00E+03 cps/ml | | | | |
| Human herpesvirus | 7538505 | 2,397,985 | 318098 | 31.81 |
| 2 5.00E+04 cps/ml | | | | |
| Human herpesvirus | 25106936 | 16,158,793 | 643599 | 64.36 |
| 2 5.00E+05 cps/ml | | | | |
| Human herpesvirus | 3724542 | 411 | 110 | 0.01 |
| 5 5.00E+00 cps/ml | | | | |
| Human herpesvirus | 4055873 | 6,745 | 1663 | 0.17 |
| 5 5.00E+01 cps/ml | | | | |
| Human herpesvirus | 5059109 | 100,061 | 19778 | 1.98 |
| 5 5.00E+02 cps/ml | | | | |
| Human herpesvirus | 5804337 | 911,127 | 156973 | 15.70 |
| 5 5.00E+03 cps/ml | | | | |
| Human herpesvirus | 14406469 | 9,498,654 | 659333 | 65.93 |
| 5 5.00E+04 cps/ml | | | | |
| Human herpesvirus | 56296526 | 45,866,190 | 814725 | 81.47 |
| 5 5.00E+05 cps/ml | | | | |
| Parvovirus B19 | 962987 | 13 | 13 | 0.00 |
| 5.00E+00 cps/ml | | | | |
| Parvovirus B19 | 1471210 | 372 | 253 | 0.03 |
| 5.00E+01 cps/ml | | | | |
| Parvovirus B19 | 1398791 | 6,786 | 4851 | 0.49 |
| 5.00E+02 cps/ml | | | | |
| Parvovirus B19 | 2390592 | 109,292 | 45718 | 4.57 |
| 5.00E+03 cps/ml | | | | |
| Parvovirus B19 | 3551088 | 1,088,973 | 306659 | 30.67 |
| 5.00E+04 cps/ml | | | | |
| Parvovirus B19 | 14983506 | 10,574,263 | 705727 | 70.57 |
| 5.00E+05 cps/ml | | | | |
| Zika virus | 2000181 | 18 | 9 | 0.00 |
| 5.00E+00 cps/ml | | | | |
| Zika virus | 2045605 | 1,636 | 800 | 0.08 |
| 5.00E+01 cps/ml | | | | |
| Zika virus | 2052384 | 15,542 | 7573 | 0.76 |
| 5.00E+02 cps/ml | | | | |
| Zika virus | 2114287 | 128,279 | 60672 | 6.07 |
| 5.00E+03 cps/ml | | | | |
| Zika virus | 4295004 | 1,611,801 | 375273 | 37.53 |
| 5.00E+04 cps/ml | | | | |
| Zika virus | 12128821 | 7,864,319 | 648399 | 64.84 |
| 5.00E+05 cps/ml | | | | |

TABLE S2
Linearity data for nasal swab tests.

| | Raw | Viral | Normalized | % viral |
|---|---|---|---|---|
| Sample Name | reads | reads | viral reads/M | reads |
| Adenovirus C | 4238057 | 153 | 36 | 0.004 |
| 5.00E−01 cps/ml | | | | |
| Adenovirus C | 4018811 | 351 | 87 | 0.009 |
| 5.00E+00 cps/ml | | | | |
| Adenovirus C | 3962966 | 3442 | 869 | 0.087 |
| 5.00E+01 cps/ml | | | | |
| Adenovirus C | 4016345 | 34039 | 8475 | 0.848 |
| 5.00E+02 cps/ml | | | | |
| Adenovirus C | 4496151 | 396848 | 88264 | 8.826 |
| 5.00E+03 cps/ml | | | | |
| Adenovirus C | 8326743 | 3382510 | 406222 | 40.622 |
| 5.00E+04 cps/ml | | | | |
| Adenovirus C | 50449122 | 39685810 | 786650 | 78.665 |
| 5.00E+05 cps/ml | | | | |
| Coronavirus 229E | 6149023 | 214 | 35 | 0.003 |
| 5.00E−01 cps/ml | | | | |
| Coronavirus 229E | 5750063 | 356 | 62 | 0.006 |
| 5.00E+00 cps/ml | | | | |
| Coronavirus 229E | 6236830 | 2688 | 431 | 0.043 |
| 5.00E+01 cps/ml | | | | |
| Coronavirus 229E | 6505667 | 24312 | 3737 | 0.374 |
| 5.00E+02 cps/ml | | | | |
| Coronavirus 229E | 5652409 | 193292 | 34196 | 3.420 |
| 5.00E+03 cps/ml | | | | |
| Coronavirus 229E | 8363014 | 2156970 | 257918 | 25.792 |
| 5.00E+04 cps/ml | | | | |
| Coronavirus 229E | 28586617 | 18738649 | 655504 | 65.550 |
| 5.00E+05 cps/ml | | | | |
| Coronavirus OC43 | 5793644 | 284 | 49 | 0.005 |
| 5.00E−01 cps/ml | | | | |
| Coronavirus OC43 | 5214864 | 169 | 32 | 0.003 |
| 5.00E+00 cps/ml | | | | |
| Coronavirus OC43 | 3719524 | 984 | 265 | 0.026 |
| 5.00E+01 cps/ml | | | | |
| Coronavirus OC43 | 5364513 | 18414 | 3433 | 0.343 |
| 5.00E+02 cps/ml | | | | |
| Coronavirus OC43 | 6556494 | 169682 | 25880 | 2.588 |
| 5.00E+03 cps/ml | | | | |
| Coronavirus OC43 | 6865095 | 1535614 | 223684 | 22.368 |
| 5.00E+04 cps/ml | | | | |
| Coronavirus OC43 | 30135365 | 20514130 | 680733 | 68.073 |
| 5.00E+05 cps/ml | | | | |
| SARS-coronavirus-2 | 5876363 | 80 | 14 | 0.0014 |
| 5.00E−01 cps/ml | | | | |
| SARS-coronavirus-2 | 5937567 | 386 | 65 | 0.0065 |
| 5.00E+00 cps/ml | | | | |
| SARS-coronavirus-2 | 5906515 | 2323 | 393 | 0.0393 |
| 5.00E+01 cps/ml | | | | |
| SARS-coronavirus-2 | 6196463 | 29116 | 4699 | 0.4699 |
| 5.00E+02 cps/ml | | | | |
| SARS-coronavirus-2 | 6337463 | 296364 | 46764 | 4.6764 |
| 5.00E+03 cps/ml | | | | |
| SARS-coronavirus-2 | 9618052 | 3123120 | 324714 | 32.4714 |
| 5.00E+04 cps/ml | | | | |
| SARS-coronavirus-2 | 38777991 | 31724265 | 818100 | 81.8100 |
| 5.00E+05 cps/ml | | | | |
| Enterovirus D68 | 532063 | 376 | 707 | 0.071 |
| 5.00E−01 cps/ml | | | | |
| Enterovirus D68 | 583519 | 1128 | 1933 | 0.193 |
| 5.00E+00 cps/ml | | | | |
| Enterovirus D68 | 525972 | 15783 | 30007 | 3.001 |
| 5.00E+01 cps/ml | | | | |
| Enterovirus D68 | 664770 | 150196 | 225937 | 22.594 |
| 5.00E+02 cps/ml | | | | |
| Enterovirus D68 | 2329323 | 1593279 | 684009 | 68.401 |
| 5.00E+03 cps/ml | | | | |
| Enterovirus D68 | 13526476 | 11683950 | 863784 | 86.378 |
| 5.00E+04 cps/ml | | | | |
| Enterovirus D68 | 61054450 | 53975801 | 884060 | 88.406 |
| 5.00E+05 cps/ml | | | | |
| Human | 6495270 | 46 | 7 | 0.00071 |
| metapneumovirus | | | | |
| 5.00E−01 cps/ml | | | | |
| Human | 5977843 | 117 | 20 | 0.00196 |
| metapneumovirus | | | | |
| 5.00E+00 cps/ml | | | | |
| Human | 6880598 | 1653 | 240 | 0.02402 |
| metapneumovirus | | | | |
| 5.00E+01 cps/ml | | | | |
| Human | 6201707 | 12874 | 2076 | 0.20759 |
| metapneumovirus | | | | |
| 5.00E+02 cps/ml | | | | |
| Human | 6964178 | 156345 | 22450 | 2.24499 |
| metapneumovirus | | | | |
| 5.00E+03 cps/ml | | | | |
| Human | 7620999 | 1092555 | 143361 | 14.33611 |
| metapneumovirus | | | | |
| 5.00E+04 cps/ml | | | | |
| Human | 12851818 | 4368361 | 339902 | 33.99022 |
| metapneumovirus | | | | |
| 5.00E+05 cps/ml | | | | |
| Respiratory | 440432 | 144 | 327 | 0.033 |
| syncytial virus | | | | |
| 5.00E−01 cps/ml | | | | |
| Respiratory | 699973 | 1177 | 1681 | 0.168 |
| syncytial virus | | | | |
| 5.00E+00 cps/ml | | | | |
| Respiratory | 704315 | 16034 | 22765 | 2.277 |
| syncytial virus | | | | |
| 5.00E+01 cps/ml | | | | |
| Respiratory | 887181 | 144108 | 162434 | 16.243 |
| syncytial virus | | | | |
| 5.00E+02 cps/ml | | | | |
| Respiratory | 2339604 | 1210105 | 517226 | 51.723 |
| syncytial virus | | | | |
| 5.00E+03 cps/ml | | | | |
| Respiratory | 8416035 | 5841399 | 694080 | 69.408 |
| syncytial virus | | | | |
| 5.00E+04 cps/ml | | | | |
| Human parainfluenza | 2222722 | 293 | 132 | 0.01 |
| virus 1 | | | | |
| 5.00E−01 cps/ml | | | | |
| Human parainfluenza | 2288154 | 1707 | 746 | 0.07 |
| virus 1 | | | | |
| 5.00E+00 cps/ml | | | | |
| Human parainfluenza | 2258275 | 23402 | 10363 | 1.04 |
| virus 1 | | | | |
| 5.00E+01 cps/ml | | | | |
| Human parainfluenza | 2432986 | 201500 | 82820 | 8.28 |
| virus 1 | | | | |
| 5.00E+02 cps/ml | | | | |
| Human parainfluenza | 4383827 | 1926719 | 439506 | 43.95 |
| virus 1 | | | | |
| 5.00E+03 cps/ml | | | | |
| Human parainfluenza | 18544950 | 13864493 | 747616 | 74.76 |
| virus 1 | | | | |
| 5.00E+04 cps/ml | | | | |
| Human parainfluenza | 66439722 | 53576332 | 806390 | 80.64 |
| virus 1 | | | | |
| 5.00E+05 cps/ml | | | | |
| Human parainfluenza | 2395804 | 90 | 38 | 0.004 |
| virus 2 | | | | |
| 5.00E−01 cps/ml | | | | |
| Human parainfluenza | 2534647 | 993 | 392 | 0.039 |
| virus 2 | | | | |
| 5.00E+00 cps/ml | | | | |
| Human parainfluenza | 2296957 | 8748 | 3809 | 0.381 |
| virus 2 | | | | |
| 5.00E+01 cps/ml | | | | |
| Human parainfluenza | 2345426 | 87545 | 37326 | 3.733 |
| virus 2 | | | | |
| 5.00E+02 cps/ml | | | | |
| Human parainfluenza | 4128042 | 1510308 | 365865 | 36.587 |
| virus 2 | | | | |
| 5.00E+03 cps/ml | | | | |
| Human parainfluenza | 11779656 | 8303687 | 704918 | 70.492 |
| virus 2 | | | | |
| 5.00E+04 cps/ml | | | | |
| Human parainfluenza | 59825974 | 50168652 | 838576 | 83.858 |
| virus 2 | | | | |
| 5.00E+05 cps/ml | | | | |
| Human parainfluenza | 2098915 | 41 | 20 | 0.002 |
| virus 3 | | | | |
| 5.00E−01 cps/ml | | | | |
| Human parainfluenza | 2030208 | 475 | 234 | 0.023 |
| virus 3 | | | | |
| 5.00E+00 cps/ml | | | | |
| Human parainfluenza | 2109806 | 4373 | 2073 | 0.207 |
| virus 3 | | | | |
| 5.00E+01 cps/ml | | | | |
| Human parainfluenza | 1876673 | 43076 | 22953 | 2.295 |
| virus 3 | | | | |
| 5.00E+02 cps/ml | | | | |
| Human parainfluenza | 2616801 | 450394 | 172116 | 17.212 |
| virus 3 | | | | |
| 5.00E+03 cps/ml | | | | |
| Human parainfluenza | 7940174 | 4886490 | 615413 | 61.541 |
| virus 3 | | | | |
| 5.00E+04 cps/ml | | | | |
| Human parainfluenza | 55218199 | 44016189 | 797132 | 79.713 |
| virus 3 | | | | |
| 5.00E+05 cps/ml | | | | |
| Influenza A virus | 4991846 | 0 | 0 | 0.0000 |
| 5.00E−01 cps/ml | | | | |
| Influenza A virus | 4953552 | 14 | 3 | 0.0003 |
| 5.00E+00 cps/ml | | | | |
| Influenza A virus | 4586147 | 1356 | 296 | 0.0296 |
| 5.00E+01 cps/ml | | | | |
| Influenza A virus | 5012641 | 13798 | 2753 | 0.2753 |
| 5.00E+02 cps/ml | | | | |
| Influenza A virus | 4687594 | 115766 | 24696 | 2.4696 |
| 5.00E+03 cps/ml | | | | |
| Influenza A virus | 6340357 | 1136217 | 179204 | 17.9204 |
| 5.00E+04 cps/ml | | | | |
| Influenza A virus | 13689607 | 8014778 | 585464 | 58.5464 |
| 5.00E+05 cps/ml | | | | |
| Influenza B virus | 5378714 | 0 | 0 | 0.000 |
| 5.00E−01 cps/ml | | | | |
| Influenza B virus | 4659922 | 45 | 10 | 0.001 |
| 5.00E+00 cps/ml | | | | |
| Influenza B virus | 7442197 | 2029 | 273 | 0.027 |
| 5.00E+01 cps/ml | | | | |
| Influenza B virus | 6033952 | 17500 | 2900 | 0.290 |
| 5.00E+02 cps/ml | | | | |
| Influenza B virus | 6448645 | 150739 | 23375 | 2.338 |
| 5.00E+03 cps/ml | | | | |
| Influenza B virus | 6658164 | 1239335 | 186138 | 18.614 |
| 5.00E+04 cps/ml | | | | |
| Influenza B virus | 18695077 | 11996473 | 641692 | 64.169 |
| 5.00E+05 cps/ml | | | | |

TABLE S3
Concordance results for clinical nasal secretion specimens.

| | Specimen | | | Viral | Genome | In-house qPCR | |
|---|---|---|---|---|---|---|---|
| # | ID | Hospital testing results | VirCapSeq results‡ | Counts | Coverage* | results # | Ct |
| 1 | CTO22- | Respiratory syncytial virus | Respiratory syncytial virus B + | 2,535,309 | 97.2% | | |
| | 15 | | | | | | |
| 2 | CTO22- | Respiratory syncytial virus | Respiratory syncytial virus A + | 1,265 | 43.7% | | |
| | 16 | | | | | | |
| 3 | CTO22- | Adenovirus | Adenovirus C + | 4,300 | 94.2% | | |
| | 17 | | | | | | |
| 4 | CTO22- | Respiratory syncytial virus | Respiratory syncytial virus A + | 69,884 | 80.7% | | |
| | 18 | | | | | | |
| 5 | CTO22- | Respiratory syncytial virus | Respiratory syncytial virus − | 0 | 0% | Respiratory | N/A |
| | 19 | | | | | virus − | |
| | | | | | | syncytial | |
| 6 | CTO22- | Respiratory syncytial virus | Respiratory syncytial virus − | 0 | 0% | Respiratory | N/A |
| | 20 | | | | | syncytial virus − | |
| 7 | CTO22- | Adenovirus | Adenovirus C +; | 3,847; 540,219 | 80.6%; 98.2% | | |
| | 21 | | Respiratory syncytial virus A + | | | | |
| 8 | CTO22- | Coronavirus OC43 | Coronavirus OC43 + | 1,123 | 40.6% | | |
| | 22 | | | | | | |
| 9 | CTO22- | Human metapneumovirus | Human metapneumovirus − | 0 | 0% | Human | N/A |
| | 23 | | | | | metapneumo- | |
| | | | | | | virus − | |
| 10 | CTO22- | Respiratory syncytial virus | Respiratory syncytial virus A + | 3,866,814 | 99.8% | | |
| | 24 | | | | | | |
| 11 | CTO22- | Adenovirus; Human parainfluenza | Adenovirus C +; | 10,522; 1,993 | 98.4%; 66.9% | | |
| | 25 | virus 3 | Human parainfluenza virus 3 + | | | | |
| 12 | CTO22- | Human metapneumovirus | Human metapneumovirus + | 6,196 | 59.7% | | |
| | 26 | | | | | | |
| 13 | CTO22- | Rhino/enterovirus | Rhino/enterovirus − | 0 | 0% | Rhino/ | N/A |
| | 27 | | | | | enterovirus − | |
| 14 | CTO22- | Adenovirus; Influenza A virus H3 | Adenovirus C+; | 3,409; 4 | 74.7%; 0% | Influenza A | N/A |
| | 28 | | Influenza A virus - | | | virus − | |
| 15 | CTO22- | Rhino/enterovirus | Human rhinovirus A + | 26,605 | 83.5% | | |
| | 29 | | | | | | |
| 16 | CTO22- | Rhino/enterovirus | Human rhinovirus C + | 24,647 | 99.2% | | |
| | 30 | | | | | | |
| 17 | CTO22- | Respiratory syncytial virus | Respiratory syncytial virus A + | 89,485 | 86.4% | | |
| | 31 | | | | | | |
| 18 | CTO22- | Rhino/enterovirus | Human rhinovirus C + | 7,065 | 99.7% | | |
| | 32 | | | | | | |
| 19 | CTO22- | Adenovirus; Influenza A virus H3 | Adenovirus −; | 0; 40,600 | 0%; 99.8% | Adenovirus − | N/A |
| | 33 | | Influenza A virus H3N2 + | | | | |
| 20 | CTO22- | Rhino/enterovirus | Human rhinovirus C + | 120,535 | 99.6% | | |
| | 34 | | | | | | |
| 21 | CTO22- | Rhino/enterovirus; Human | Human rhinovirus C +; | 698; 0 | 48%; 0% | Human | N/A |
| | 35 | parainfluenza virus 3 | Human arainfluenza virus 3 − | | | parainfluenza | |
| | | | | | | virus 3 − | |
| 22 | CTO22- | Rhino/enterovirus | Human rhinovirus C + | 112,239 | 99.7% | | |
| | 36 | | | | | | |
| 23 | CTO22- | Rhino/enterovirus | Human rhinovirus A + | 224,119 | 99.9% | | |
| | 37 | | | | | | |
| 24 | CTO22- | Adenovirus | Adenovirus F + | 626,893 | 99.6% | | |
| | 38 | | | | | | |
| 25 | CTO22- | Adenovirus | Adenovirus C + | 116 | 13.7% | | |
| | 39 | | | | | | |
| 26 | CTO22- | Human parainfluenza virus 3 | Human parainfluenza virus 3 + | 945,440 | 99.8% | | |
| | 40 | | | | | | |
| 27 | CTO22- | Adenovirus; Respiratory syncytial | Adenovirus −; Respiratory syncytial | 0; 5,592 | 0%; 59.9% | Adenovirus − | N/A |
| | 41 | virus | virus A + | | | | |
| 28 | CTO22- | Rhino/enterovirus | Human rhinovirus C + | 168,807 | 99.6% | SARS- | |
| | 42 | | | | | | |
| 29 | CTO22- | Adenovirus; SARS-coronavirus-2 | Adenovirus C +; | 646,895; 0 | 99.5%; 0% | coronavirus-2 − | N/A |
| | 43 | | SARS-coronavirus-2 − | | | | |
| 30 | CTO22- | Influenza A virus H3 | Influenza A virus H3N2 + | 4,221,763 | 100% | | |
| | 44 | | | | | | |
| 31 | CTO22- | Adenovirus | Adenovirus C + | 144 | 25.3% | | |
| | 45 | | | | | | |
| 32 | CTO22- | Adenovirus | Adenovirus − | 7 | 0% | Adenovirus + | 36.5 |
| | 46 | | | | | | |
| 33 | CTO22- | Respiratory syncytial virus | Respiratory syncytial virus − | 0 | 0% | RSV − | N/A |
| | 47 | | | | | | |
| 34 | CTO22- | Influenza A virus H3 | Influenza A virus H3N2 + | 753,232 | 100% | | |
| | 48 | | | | | | |
| 35 | CTO22- | Respiratory syncytial virus | Respiratory syncytial virus A + | 81,103 | 94.7% | | |
| | 49 | | | | | | |
| 36 | CTO22- | Rhino/enterovirus | Human rhinovirus B + | 1,456,287 | 99.9% | | |
| | 50 | | | | | | |
| 37 | CTO22- | Adenovirus; | Adenovirus C +; | 304; 1,280 | 37.4%; 52.1% | | |
| | 51 | Human metapneumovirus | Human metapneumovirus + | | | | |
| 38 | CTO22- | Respiratory syncytial virus | Respiratory syncytial virus A + | 26,648 | 99.9% | | |
| | 52 | | | | | | |
| 39 | CTO22- | Respiratory syncytial virus | Respiratory syncytial virus A + | 146,004 | 99.9% | | |
| | 53 | | | | | | |
| 40 | CTO22- | Coronavirus OC43 | Coronavirus OC43 +; | 678; 649 | 14%; 40.9% | | |
| | 54 | | Influenza C virus + | | | | |
| 41 | CTO22- | Human parainfluenza virus 3 | Human parainfluenza virus 3 + | 5222 | 90% | | |
| | 55 | | | | | | |
| 42 | CTO22- | Respiratory syncytial virus | Respiratory syncytial virus B + | 84,352 | 99.6% | | |
| | 56 | | | | | | |
| 43 | CTO22- | Human parainfluenza virus 3; | Human parainfluenza virus 3 +; | 1,123; 2,087 | 47.3%; 67.2% | | |
| | 57 | Respiratory syncytial virus | Respiratory syncytial virus A + | | | | |
| 44 | CTO22- | Coronavirus OC43 | Coronavirus OC43 + | 934 | 43.3% | | |
| | 58 | | | | | | |
| 45 | CTO22- | Human metapneumovirus | Human metapneumovirus + | 971,870 | 99.9% | | |
| | 59 | | | | | | |
| 46 | CTO22- | Rhino/enterovirus | Enterovirus D68 + | 17,498 | 99.1% | | |
| | 60 | | | | | | |
| 47 | CTO22- | Influenza A virus H3 | Influenza A virus H3N2 + | 203 | 51.7% | | |
| | 61 | | | | | | |
| 48 | CTO22- | Respiratory syncytial virus | Respiratory syncytial virus B + | 404,015 | 99.8% | | |
| | 62 | | | | | | |
| 49 | CTO22- | Rhino/enterovirus | Human rhinovirus C + | 897,851 | 100% | | |
| | 63 | | | | | | |
| 50 | CTO2 | Rhino/enterovirus | Human rhinovirus C + | 3,671,229 | 100% | | |
| | 2-64 | | | | | | |

‡Sequencing results: +, positive; −, negative
*Influenza genome coverage reported for reads mapped against concatenated full genomes.
# Results for in-house qPCR done on specimen that were negative by VirCapSeq analysis; +, qPCR positive (Ct < 40); −, qPCR negative (N/A, Ct > 40)

### Example 2—Summary of Advantage and Explanation of Differential Clustering Strategy Used

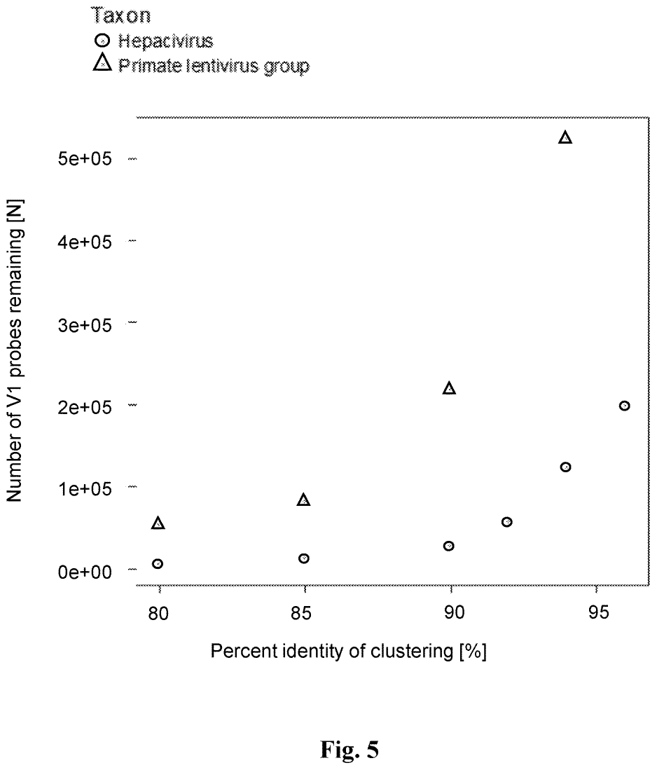

Clustering values were deduced from graphs displaying the total number of probes selected for viral taxa vs. clustering percentage (see FIG. 5). Differential clustering for hepaciviruses reduced probe number for Flaviviridae by ~100,000, and differential clustering for human lentiviruses reduced probe number for Retroviridae by ~700,000.

Clustering values were chosen where reduction in percent identity resulted in only marginal reduction in probe number, provided that total identity did not become prohibitive of hybridization (not <80% sequence identity).

Additional reduction in the number of probes resulted from altered probe length and spacing.

TABLE 6
Example of alterations in probe library design strategy
and their effects on the VirCapSeq-VERT platform:

| | Version A | Version B |
|---|---|---|
| Probe number | 1,990,000 | 880,000 |
| Probe length | 50-100 nucleotides | 120 nucleotides |
| Probe spacing | 50-150 nucleotides | 60 nucleotides |
| Probe clustering | Probes at 98% | Sequences for probe selection |
| | | at 96% for all taxa |
| | | Selected probes for |
| | | hepaciviruses at 92%* |
| | | Selected probes for primate |
| | | lentiviruses at 85%^{#} |

*Differential clustering for hepaciviruses reduced probe number for Flaviviridae by ~100,000
^{#}Differential clustering for human lentiviruses reduced probe number for Retroviridae by ~700,000

### Example 3—a Novel Virome-Capture-Sequencing (VirCapSeq) Method for Viral Diagnosis and Virus Discovery

The implementation of high throughput nucleic acid sequencing in detection and differential diagnosis of viral infections in research and clinical laboratories has proven difficult. Described herein is an improved and redesigned virome-capture-sequencing platform for vertebrate viruses (VirCapSeq-VERT) that increases the sensitivity of sequence-based virus detection and characterization. Features of the VirCapSeq-VERT platform include:
A near 50% reduction in the number of probes required to capture the vertebrate virome;
A selection of probes that ensures a more even coverage of viral taxa in the probe set;
Longer probes spaced at longer intervals;
Probes which can detect of recent viral sequences.

The features of the platform described herein reduce the cost of its synthesis and enhance its performance. The VirCapSeq-VERT platform described herein also increase the feasibility of designing a panmicrobial platform for diagnosis and surveillance of all infectious diseases by adding probes for other microbes (e.g., bacteria, fungi, and parasites).

Specifically, the VirCapSeq-VERT platform described herein uses probes spanning the genomes of members of all virus taxa known to infect vertebrates, including humans. This platform requires about 1 million fewer probes than other versions while also including probes representing new viral sequences. The platform also comprises longer probes that enable greater capture efficiency for more distantly related viruses. The probe length may be up to 300 nucleotides, as supported by experimental data for probes at least 100 nucleotides in length and in silico data suggesting that even longer probes will perform similarly. Indeed, the VirCapSeq-VERT platform described herein has been shown to span all vertebrate virus taxa using only 879,173 probes.

The VirCapSeq-VERT platform described herein may also include probes for the common set of external RNA controls developed by the External RNA Controls Consortium (ERCC). Specifically, for half of the ERCC sequences, there may be probes and for half there may be no probes. This enables users to validate the efficacy of enrichment using spiked ERCC RNA. From these probes, a biotinylated oligonucleotide library is used for solution-based capture of virus nucleic acids present in complex samples containing variable proportions of different virus and host nucleic acids. Our experimental data confirm that the VirCapSeq-VERT platform results in a 1000-10,000-fold increase in viral reads from a wide range of sample types including blood, respiratory secretions, saliva, cerebrospinal fluid, urine, tissue homogenates and wastewater when compared to conventional Illumina sequencing using established virus enrichment procedures, including filtration, nuclease treatments, and rRNA subtraction.

Thus, the VirCapSeq-VERT platform described herein improves the transition to high-throughput sequencing in clinical diagnostics by enabling in-depth virome analyses of samples with a streamlined and more efficient capture system. The platform allows for more sensitive and economic vertebrate virus sequence detection and determination in clinical specimens or environmental samples, while also having the potential to be merged with capture systems targeting other pathogens.

TABLE A
Examples of virus taxa for VirCapSeq-VERT platform design

| | | Parent | Parent |
|---|---|---|---|
| Name | tax_id | Name | tax_id |
| Adenoviridae | 10508 | dsDNA viruses, | 35237 |
| | | no RNA stage | |
| Alloherpesviridae | 548682 | Herpesvirales | 548681 |
| Alphacoronavirus | 693996 | Coronavirinae | 693995 |
| Alphaherpesvirinae | 10293 | Herpesviridae | 10292 |
| Alphanodavirus | 143920 | Nodaviridae | 12283 |
| Alphapapillomavirus | 333750 | Papillomaviridae | 151340 |
| Alphapermutotetravirus | 1283211 | Permutotetraviridae | 1283210 |
| Alpharetrovirus | 153057 | Orthoretrovirinae | 327045 |
| Alphatorquevirus | 687331 | Anelloviridae | 687329 |
| Alphavirus | 11019 | Togaviridae | 11018 |
| Amdoparvovirus | 310911 | Parvovirinae | 40119 |
| Anelloviridae | 687329 | ssDNA viruses | 29258 |
| Aphthovirus | 12109 | Picornaviridae | 12058 |
| Aquabirnavirus | 39750 | Birnaviridae | 10993 |
| Aquamavirus | 1330065 | Picornaviridae | 12058 |
| Aquaparamyxovirus | 1232658 | Paramyxovirinae | 11159 |
| Aquareovirus | 10979 | Spinareovirinae | 689831 |
| Arenaviridae | 11617 | ssRNA negative- | 35301 |
| | | strand viruses | |
| Arenavirus | 11618 | Arenaviridae | 11617 |
| Arteriviridae | 76803 | Nidovirales | 76804 |
| Arterivirus | 11046 | Arteriviridae | 76803 |
| Asfarviridae | 137992 | dsDNA viruses, | 35237 |
| | | no RNA stage | |
| Asfivirus | 39743 | Asfarviridae | 137992 |
| Astroviridae | 39733 | ssRNA positive- | 35278 |
| | | strand viruses, | |
| | | no DNA stage | |
| Atadenovirus | 100953 | Adenoviridae | 10508 |
| Aurivirus | 1513230 | Malacoherpesviridae | 548685 |
| Avastrovirus | 249589 | Astroviridae | 39733 |
| Aveparvovirus | 1511864 | Parvovirinae | 40119 |
| Aviadenovirus | 10552 | Adenoviridae | 10508 |
| Avibirnavirus | 39751 | Birnaviridae | 10993 |
| Avihepadnavirus | 10437 | Hepadnaviridae | 10404 |
| Avihepatovirus | 691955 | Picornaviridae | 12058 |
| Avipoxvirus | 10260 | Chordopoxvirinae | 10241 |
| Avisivirus | 1511771 | Picornaviridae | 12058 |
| Avulavirus | 260963 | Paramyxovirinae | 11159 |
| Bafinivirus | 694018 | Torovirinae | 694017 |
| Batrachovirus | 692605 | Alloherpesviridae | 548682 |
| Betacoronavirus | 694002 | Coronavirinae | 693995 |
| Betaherpesvirinae | 10357 | Herpesviridae | 10292 |
| Betanodavirus | 143919 | Nodaviridae | 12283 |
| Betapapillomavirus | 333922 | Papillomaviridae | 151340 |
| Betaretrovirus | 140052 | Orthoretrovirinae | 327045 |
| Betatorquevirus | 687332 | Anelloviridae | 687329 |
| Birnaviridae | 10993 | dsRNA viruses | 35325 |
| Blosnavirus | 564643 | Birnaviridae | 10993 |
| Bocaparvovirus | 1507401 | Parvovirinae | 40119 |
| Bornaviridae | 178830 | Mononegavirales | 11157 |
| Bornavirus | 186458 | Bornaviridae | 178830 |
| Bracorhabdovirus | 490109 | unclassified | 35303 |
| | | Rhabdoviridae | |
| Bunyaviridae | 11571 | ssRNA negative- | 35301 |
| | | strand viruses | |
| Caliciviridae | 11974 | ssRNA positive- | 35278 |
| | | strand viruses, | |
| | | no DNA stage | |
| Capripoxvirus | 10265 | Chordopoxvirinae | 10241 |
| Cardiovirus | 12103 | Picornaviridae | 12058 |
| Cervidpoxvirus | 573055 | Chordopoxvirinae | 10241 |
| Chipapillomavirus | 934800 | Papillomaviridae | 151340 |
| Chloriridovirus | 10491 | Iridoviridae | 10486 |
| Chordopoxvirinae | 10241 | Poxviridae | 10240 |
| Circoviridae | 39724 | ssDNA viruses | 29258 |
| Circovirus | 39725 | Circoviridae | 39724 |
| Coltivirus | 10911 | Spinareovirinae | 689831 |
| Copiparvovirus | 1511888 | Parvovirinae | 40119 |
| Coronaviridae | 11118 | Nidovirales | 76804 |
| Coronavirinae | 693995 | Coronaviridae | 11118 |
| Cosavirus | 586418 | Picornaviridae | 12058 |
| Crocodylidpoxvirus | 1285599 | Chordopoxvirinae | 10241 |
| Cuevavirus | 1513236 | Filoviridae | 11266 |
| Cyprinivirus | 692606 | Alloherpesviridae | 548682 |
| Cytomegalovirus | 10358 | Betaherpesvirinae | 10357 |
| Cytorhabdovirus | 11305 | Rhabdoviridae | 11270 |
| Deltacoronavirus | 1159901 | Coronavirinae | 693995 |
| Deltapapillomavirus | 325454 | Papillomaviridae | 151340 |
| Deltaretrovirus | 153136 | Orthoretrovirinae | 327045 |
| Deltatorquevirus | 687334 | Anelloviridae | 687329 |
| Deltavirus | 39759 | Viruses | 10239 |
| Dengue virus group | 11052 | Flavivirus | 11051 |
| Densovirinae | 40120 | Parvoviridae | 10780 |
| Dependoparvovirus | 10803 | Parvovirinae | 40119 |
| Dicipivirus | 1330067 | Picornaviridae | 12058 |
| Dinornavirus | 674976 | Alvernaviridae | 866787 |
| Dyodeltapapillomavirus | 936056 | Papillomaviridae | 151340 |
| Dyoepsilonpapillomavirus | 935646 | Papillomaviridae | 151340 |
| Dyoetapapillomavirus | 935641 | Papillomaviridae | 151340 |
| Dyoiotapapillomavirus | 934804 | Papillomaviridae | 151340 |
| Dyokappapapillomavirus | 1513238 | Papillomaviridae | 151340 |
| Dyolambdapapillomavirus | 1513239 | Papillomaviridae | 151340 |
| Dyomupapillomavirus | 1513240 | Papillomaviridae | 151340 |
| Dyonupapillomavirus | 1513241 | Papillomaviridae | 151340 |
| Dyoomikronpapillomavirus | 1513242 | Papillomaviridae | 151340 |
| Dyopipapillomavirus | 1513243 | Papillomaviridae | 151340 |
| Dyorhopapillomavirus | 1513244 | Papillomaviridae | 151340 |
| Dyosigmapapillomavirus | 1513245 | Papillomaviridae | 151340 |
| Dyothetapapillomavirus | 1052159 | Papillomaviridae | 151340 |
| Dyoxipapillomavirus | 1513246 | Papillomaviridae | 151340 |
| Dyozetapapillomavirus | 934803 | Papillomaviridae | 151340 |
| Ebolavirus | 186536 | Filoviridae | 11266 |
| Enterovirus | 12059 | Picornaviridae | 12058 |
| Entomopoxvirinae | 10284 | Poxviridae | 10240 |
| Ephemerovirus | 32613 | Rhabdoviridae | 11270 |
| Epsilonretrovirus | 153137 | Orthoretrovirinae | 327045 |
| Epsilontorquevirus | 687335 | Anelloviridae | 687329 |
| Equine | 11654 | Lentivirus | 11646 |
| lentivirus group | | | |
| Erbovirus | 194961 | Picornaviridae | 12058 |
| Erythroparvovirus | 40121 | Parvovirinae | 40119 |
| Etapapillomavirus | 325458 | Papillomaviridae | 151340 |
| Etatorquevirus | 687337 | Anelloviridae | 687329 |
| Ferlavirus | 1283308 | Paramyxovirinae | 11159 |
| Filoviridae | 11266 | Mononegavirales | 11157 |
| Flaviviridae | 11050 | ssRNA positive- | 35278 |
| | | strand viruses, | |
| | | no DNA stage | |
| Flavivirus | 11051 | Flaviviridae | 11050 |
| Gallivirus | 1511775 | Picornaviridae | 12058 |
| Gammacoronavirus | 694013 | Coronavirinae | 693995 |
| Gammaherpesvirinae | 10374 | Herpesviridae | 10292 |
| Gammapapillomavirus | 325455 | Papillomaviridae | 151340 |
| Gammaretrovirus | 153135 | Orthoretrovirinae | 327045 |
| Gammatorquevirus | 687333 | Anelloviridae | 687329 |
| Gyrovirus | 227307 | Circoviridae | 39724 |
| Hantavirus | 11598 | Bunyaviridae | 11571 |
| Henipavirus | 260964 | Paramyxovirinae | 11159 |
| Hepacivirus | 11102 | Flaviviridae | 11050 |
| Hepadnaviridae | 10404 | Retro-transcribing | 35268 |
| | | viruses | |
| Hepatovirus | 12091 | Picornaviridae | 12058 |
| Hepeviridae | 291484 | ssRNA positive- | 35278 |
| | | strand viruses, | |
| | | no DNA stage | |
| Hepevirus | 186677 | Hepeviridae | 291484 |
| Herpesvirales | 548681 | dsDNA viruses, | 35237 |
| | | no RNA stage | |
| Herpesviridae | 10292 | Herpesvirales | 548681 |
| Hunnivirus | 1431456 | Picornaviridae | 12058 |
| Ichtadenovirus | 691957 | Adenoviridae | 10508 |
| Ictalurivirus | 172653 | Alloherpesviridae | 548682 |
| Iltovirus | 180255 | Alphaherpesvirinae | 10293 |
| Influenzavirus D | 1511083 | unclassified | 35324 |
| | | Orthomyxoviridae | |
| Intracisternal | 11749 | unclassified | 35276 |
| A-particles | | Retroviridae | |
| Iotatorquevirus | 687339 | Anelloviridae | 687329 |
| Iridoviridae | 10486 | dsDNA viruses, | 35237 |
| | | no RNA stage | |
| Iridovirus | 10487 | Iridoviridae | 10486 |
| Isavirus | 324913 | Orthomyxoviridae | 11308 |
| Japanese | 11071 | Flavivirus | 11051 |
| encephalitis | | | |
| virus group | | | |
| Kappapapillomavirus | 325457 | Papillomaviridae | 151340 |
| Kappatorquevirus | 1218487 | Anelloviridae | 687329 |
| Kobuvirus | 194960 | Picornaviridae | 12058 |
| Kokobera | 303179 | Flavivirus | 11051 |
| virus group | | | |
| Lagovirus | 95339 | Caliciviridae | 11974 |
| Lambdapapillomavirus | 325462 | Papillomaviridae | 151340 |
| Lambdatorquevirus | 1218489 | Anelloviridae | 687329 |
| Lentivirus | 11646 | Orthoretrovirinae | 327045 |
| Leporipoxvirus | 10270 | Chordopoxvirinae | 10241 |
| Lymphocryptovirus | 10375 | Gammaherpesvirinae | 10374 |
| Lymphocystivirus | 10494 | Iridoviridae | 10486 |
| Lyssavirus | 11286 | Rhabdoviridae | 11270 |
| Macavirus | 548687 | Gammaherpesvirinae | 10374 |
| Malacoherpesviridae | 548685 | Herpesvirales | 548681 |
| Mamastrovirus | 249588 | Astroviridae | 39733 |
| Marburgvirus | 186537 | Filoviridae | 11266 |
| Mardivirus | 180252 | Alphaherpesvirinae | 10293 |
| Mastadenovirus | 10509 | Adenoviridae | 10508 |
| Megalocytivirus | 308906 | Iridoviridae | 10486 |
| Megrivirus | 1330069 | Picornaviridae | 12058 |
| Metapneumovirus | 162387 | Pneumovirinae | 11244 |
| Mischivirus | 1511778 | Picornaviridae | 12058 |
| Modoc virus group | 29260 | Flavivirus | 11051 |
| Molluscipoxvirus | 10278 | Chordopoxvirinae | 10241 |
| Mononegavirales | 11157 | ssRNA negative- | 35301 |
| | | strand viruses | |
| Morbillivirus | 11229 | Paramyxovirinae | 11159 |
| Mosavirus | 1481451 | Picornaviridae | 12058 |
| mosquito-borne viruses | 59562 | Flavivirus | 11051 |
| Mupapillomavirus | 334202 | Papillomaviridae | 151340 |
| Muromegalovirus | 10365 | Betaherpesvirinae | 10357 |
| Nairovirus | 11592 | Bunyaviridae | 11571 |
| Nebovirus | 696855 | Caliciviridae | 11974 |
| Negevirus | 1307798 | unclassified ssRNA | 38173 |
| | | positive-strand | |
| | | viruses | |
| Nidovirales | 76804 | ssRNA positive- | 35278 |
| | | strand viruses, | |
| | | no DNA stage | |
| Nodaviridae | 12283 | ssRNA positive- | 35278 |
| | | strand viruses, | |
| | | no DNA stage | |
| Norovirus | 142786 | Caliciviridae | 11974 |
| Novirhabdovirus | 186778 | Rhabdoviridae | 11270 |
| Ntaya virus group | 29261 | Flavivirus | 11051 |
| Nucleorhabdovirus | 11306 | Rhabdoviridae | 11270 |
| Nupapillomavirus | 475861 | Papillomaviridae | 151340 |
| Nyamiviridae | 1513294 | Mononegavirales | 11157 |
| Nyavirus | 1513295 | Nyamiviridae | 1513294 |
| Omegapapillomavirus | 936061 | Papillomaviridae | 151340 |
| Orbivirus | 10892 | Sedoreovirinae | 689832 |
| Orthobunyavirus | 11572 | Bunyaviridae | 11571 |
| Orthohepadnavirus | 10405 | Hepadnaviridae | 10404 |
| Orthomyxoviridae | 11308 | ssRNA negative- | 35301 |
| | | strand viruses | |
| Orthopoxvirus | 10242 | Chordopoxvirinae | 10241 |
| Orthoreovirus | 10882 | Spinareovirinae | 689831 |
| Orthoretrovirinae | 327045 | Retroviridae | 11632 |
| Oscivirus | 1511780 | Picornaviridae | 12058 |
| Ostreavirus | 548686 | Malacoherpesviridae | 548685 |
| Papillomaviridae | 151340 | dsDNA viruses, | 35237 |
| | | no RNA stage | |
| Paramyxoviridae | 11158 | Mononegavirales | 11157 |
| Paramyxovirinae | 11159 | Paramyxoviridae | 11158 |
| Parapoxvirus | 10257 | Chordopoxvirinae | 10241 |
| Parechovirus | 138954 | Picornaviridae | 12058 |
| Parvoviridae | 10780 | ssDNA viruses | 29258 |
| Parvovirinae | 40119 | Parvoviridae | 10780 |
| Pasivirus | 1511782 | Picornaviridae | 12058 |
| Passerivirus | 1511802 | Picornaviridae | 12058 |
| Pegivirus | 1307799 | Flaviviridae | 11050 |
| Percavirus | 548688 | Gammaherpesvirinae | 10374 |
| Perhabdovirus | 1298653 | Rhabdoviridae | 11270 |
| Pestivirus | 11095 | Flaviviridae | 11050 |
| Phipapillomavirus | 934802 | Papillomaviridae | 151340 |
| Phlebovirus | 11584 | Bunyaviridae | 11571 |
| Picobirnaviridae | 585893 | dsRNA viruses | 35325 |
| Picobirnavirus | 104394 | Picobirnaviridae | 585893 |
| Picornavirales | 464095 | ssRNA positive- | 35278 |
| | | strand viruses, | |
| | | no DNA stage | |
| Picornaviridae | 12058 | Picornavirales | 464095 |
| Pipapillomavirus | 334211 | Papillomaviridae | 151340 |
| Pneumovirinae | 11244 | Paramyxoviridae | 11158 |
| Pneumovirus | 11245 | Pneumovirinae | 11244 |
| Polyomaviridae | 151341 | dsDNA viruses, | 35237 |
| | | no RNA stage | |
| Polyomavirus | 10624 | Polyomaviridae | 151341 |
| Poxviridae | 10240 | dsDNA viruses, | 35237 |
| | | no RNA stage | |
| Proboscivirus | 548689 | Betaherpesvirinae | 10357 |
| Protoparvovirus | 1506574 | Parvovirinae | 40119 |
| Psipapillomavirus | 935650 | Papillomaviridae | 151340 |
| Quadrivirus | 1299297 | Quadriviridae | 1299296 |
| Quaranjavirus | 1299308 | Orthomyxoviridae | 11308 |
| Ranavirus | 10492 | Iridoviridae | 10486 |
| Recovirus | 873551 | Caliciviridae | 11974 |
| Reoviridae | 10880 | dsRNA viruses | 35325 |
| Respirovirus | 186938 | Paramyxovirinae | 11159 |
| Retroviridae | 11632 | Retro-transcribing | 35268 |
| | | viruses | |
| Rhabdoviridae | 11270 | Mononegavirales | 11157 |
| Rhadinovirus | 10379 | Gammaherpesvirinae | 10374 |
| Rhopapillomavirus | 936057 | Papillomaviridae | 151340 |
| Rio Bravo | 29262 | Flavivirus | 11051 |
| virus group | | | |
| Rosavirus | 1511804 | Picornaviridae | 12058 |
| Roseolovirus | 40272 | Betaherpesvirinae | 10357 |
| Rotavirus | 10912 | Sedoreovirinae | 689832 |
| Rubivirus | 11040 | Togaviridae | 11018 |
| Rubulavirus | 39744 | Paramyxovirinae | 11159 |
| Salivirus | 688449 | Picornaviridae | 12058 |
| Salmonivirus | 692607 | Alloherpesviridae | 548682 |
| Sapelovirus | 686982 | Picornaviridae | 12058 |
| Sapovirus | 95341 | Caliciviridae | 11974 |
| Scutavirus | 1232637 | Alphaherpesvirinae | 10293 |
| Seaborne | 29264 | Flavivirus | 11051 |
| tick-borne | | | |
| virus group | | | |
| Seadornavirus | 208294 | Sedoreovirinae | 689832 |
| Sedoreovirinae | 689832 | Reoviridae | 10880 |
| Senecavirus | 586425 | Picornaviridae | 12058 |
| Siadenovirus | 129876 | Adenoviridae | 10508 |
| Sigmapapillomavirus | 935635 | Papillomaviridae | 151340 |
| Sigmavirus | 1308858 | Rhabdoviridae | 11270 |
| Simplexvirus | 10294 | Alphaherpesvirinae | 10293 |
| Spinareovirinae | 689831 | Reoviridae | 10880 |
| Sprivivirus | 1513299 | Rhabdoviridae | 11270 |
| Spumaretrovirinae | 327046 | Retroviridae | 11632 |
| Spumavirus | 11640 | Spumaretrovirinae | 327046 |
| Suipoxvirus | 10275 | Chordopoxvirinae | 10241 |
| Taupapillomavirus | 934799 | Papillomaviridae | 151340 |
| Teschovirus | 118139 | Picornaviridae | 12058 |
| Tetraparvovirus | 1511911 | Parvovirinae | 40119 |
| Thetapapillomavirus | 334213 | Papillomaviridae | 151340 |
| Thetatorquevirus | 687338 | Anelloviridae | 687329 |
| Thogotovirus | 35323 | Orthomyxoviridae | 11308 |
| Tibrovirus | 1299306 | Rhabdoviridae | 11270 |
| tick-borne | 29263 | Flavivirus | 11051 |
| encephalitis | | | |
| virus group | | | |
| Togaviridae | 11018 | ssRNA positive- | 35278 |
| | | strand viruses, | |
| | | no DNA stage | |
| Torovirinae | 694017 | Coronaviridae | 11118 |
| Torovirus | 11155 | Torovirinae | 694017 |
| Tremovirus | 689759 | Picornaviridae | 12058 |
| Tupavirus | 1513300 | Rhabdoviridae | 11270 |
| Upsilonpapillomavirus | 936058 | Papillomaviridae | 151340 |
| Varicellovirus | 10319 | Alphaherpesvirinae | 10293 |
| Vesiculovirus | 11271 | Rhabdoviridae | 11270 |
| Vesivirus | 95337 | Caliciviridae | 11974 |
| Yatapoxvirus | 10282 | Chordopoxvirinae | 10241 |
| Yellow fever | 40005 | Flavivirus | 11051 |
| virus group | | | |
| Zetapapillomavirus | 333918 | Papillomaviridae | 151340 |
| Zetatorquevirus | 687336 | Anelloviridae | 687329 |

### REFERENCES

1. Girum T, Lentiro K, Geremew M, Migora B, Shewamare S, Shimbre M S. 2021. Optimal strategies for COVID-19 prevention from global evidence achieved through social distancing, stay at home, travel restriction and lockdown: a systematic review. Arch Public Health 79:150.
2. Guan W J, Chen R C, Zhong N S. 2020. Strategies for the prevention and management of coronavirus disease 2019. Eur Respir J 55.
3. Hakim M S, Widyaningsih S A. 2023. The recent re-emergence of human monkeypox: Would it become endemic beyond Africa? J Infect Public Health 16:332-340.
4. Guan H, Gul I, Xiao C, Ma S, Liang Y, Yu D, Liu Y, Liu H, Zhang C Y, Li J, Qin P. 2023. Emergence, phylogeography, and adaptive evolution of mpox virus. New Microbes New Infect 52:101102.
5. Pallansch M A. 2022. Circulating Poliovirus in New York-New Instance of an Old Problem. N Engl

### 301 J Med 387:1725-1728.

6. Rai A, Uwishema O, Uweis L, El Saleh R, Arab S, Abbass M, Wellington J, Musabirema F, Adanur I, Patrick Onyeaka C V. 2022. Polio returns to the USA: An epidemiological alert. Ann Med Surg (Lond) 82:104563.
7. Benschop K S, Albert J, Anton A, Andres C, Aranzamendi M, Armannsdottir B, Bailly J L, Baldanti F, Baldvinsdottir G E, Beard S, Berginc N, Bottcher S, Blomqvist S, Bubba L, Calvo C, Cabrerizo M, Cavallero A, Celma C, Ceriotti F, Costa I, Cottrell S, Del Cuerpo M, Dean J, Dembinski J L, Diedrich S, Diez-Domingo J, Dorenberg D, Duizer E, Dyrdak R, Fanti D, Farkas A, Feeney S, Flipse J, De Gascun C, Galli C, Georgieva I, Gifford L, Guiomar R, Honemann M, Ikonen N, Jeannoel M, Josset L, Keeren K, Lopez-Labrador F X, Maier M, McKenna J, Meijer A, Mengual-Chulia B, Midgley S E, Mirand A, et al. 2021. Re-emergence of enterovirus D68 in Europe after easing the COVID-19 lockdown, September 2021. Euro Surveill 26.
8. Shah M M, Perez A, Lively J Y, Avadhanula V, Boom J A, Chappell J, Englund J A, Fregoe W, Halasa N B, Harrison C J, Hickey R W, Klein E J, McNeal M M, Michaels M G, Moffatt M E, Otten C, Sahni L C, Schlaudecker E, Schuster J E, Selvarangan R, Staat M A, Stewart L S, Weinberg G A, Williams J V, Ng T F F, Routh J A, Gerber S I, McMorrow M L, Rha B, Midgley C M. 2021. Enterovirus D68-Associated Acute Respiratory Illness horizontal line New Vaccine Surveillance Network, United States, July-November 2018-2020. MMWR Morb Mortal Wkly Rep 70:1623-1628.
9. Pruccoli G, Castagno E, Raffaldi I, Denina M, Barisone E, Baroero L, Timeus F, Rabbone I, Monzani A, Terragni G M, Lovera C, Brach Del Prever A, Manzoni P, Barbaglia M, Roasio L, De Franco S, Calitri C, Lupica M, Felici E, Marciano C, Santovito S, Militerno G, Abrigo E, Curtoni A, Quarello P, Bondone C, Garazzino S. 2023. The Importance of RSV Epidemiological Surveillance: A Multicenter Observational Study of RSV Infection during the COVID-19 Pandemic. Viruses 15.
10. Ghosh A, Annigeri S, Hemram S K, Dey P K, Mazumder S. 2022. Clinico-demographic Profile and Predictors of Intensive Care Need in Children with Respiratory Syncytial Virus-associated Acute Lower Respiratory Illness during Its Recent Outbreak alongside Ongoing COVID-19 Pandemic: An Eastern Indian Perspective. Indian J Crit Care Med 26:1210-1217.
11. Rana M S, Alam M M, Ikram A, Salman M, Mere M O, Usman M, Umair M, Zaidi S S Z, Arshad Y. 2021. Emergence of measles during the COVID-19 pandemic threatens Pakistan's children and the wider region. Nat Med 27:1127-1128.
12. Abbasi J. 2023. Amid Ohio Measles Outbreak, New Global Report Warns of Decreased Vaccination During COVID-19 Pandemic. JAMA 329:9-11.
13. WHO. 1996. Infectious diseases kill over 17 million people a year: WHO warns of global crisis. World Health Organization News Room.
14. Fonkwo P N. 2008. Pricing infectious disease. The economic and health implications of infectious
   336 diseases. EMBO Rep 9 Suppl 1: S13-7.
15. Schlaberg R, Chiu C Y, Miller S, Procop G W, Weinstock G, Professional Practice C, Committee on Laboratory Practices of the American Society for M, Microbiology Resource Committee of the College of American P. 2017. Validation of Metagenomic Next-Generation Sequencing Tests for Universal Pathogen Detection. Arch Pathol Lab Med 141:776-786.
16. Briese T, Kapoor A, Mishra N, Jain K, Kumar A, Jabado O J, Lipkin W I. 2015. Virome Capture Sequencing Enables Sensitive Viral Diagnosis and Comprehensive Virome Analysis. mBio 6:e01491-15.
17. Jarvie T. 2005. Next generation sequencing technologies. Drug Discov Today Technol 2:255-60.
18. Anderson M E, Nagy-Szakal D, Jain K, Patrone C C, Frattini M G, Lipkin W I, Geskin L J. 2018. Highly Sensitive Virome Capture Sequencing Technique VirCapSeq-VERT Identifies Partial Noncoding Sequences but no Active Viral Infection in Cutaneous T-Cell Lymphoma. J Invest Dermatol 138:1671-1673.
19. Cummings M J, Tokarz R, Bakamutumaho B, Kayiwa J, Byaruhanga T, Owor N, Namagambo B, Wolf A, Mathema B, Lutwama J J, Schluger N W, Lipkin W I, O'Donnell M R. 2019. Precision Surveillance for Viral Respiratory Pathogens: Virome Capture Sequencing for the Detection and Genomic Characterization of Severe Acute Respiratory Infection in Uganda. Clin Infect Dis 68:1118-1125.
20. Dugue R, Cay-Martinez K C, Thakur K T, Garcia J A, Chauhan L V, Williams S H, Briese T, Jain K, Foca M, McBrian D K, Bain J M, Lipkin W I, Mishra N. 2020. Neurologic manifestations in an infant with COVID-19. Neurology 94:1100-1102.
21. Franke N, Bette M, Marquardt A, Briese T, Lipkin W I, Kurz C, Ehrenreich J, Mack E, Baying B, Benes V, Rodepeter F R, Neff A, Teymoortash A, Eivazi B, Geisthoff U, Stuck B A, Bakowsky U, Mandic R. 2018. Virome Analysis Reveals No Association of Head and Neck Vascular Anomalies with an Active Viral Infection. In Vivo 32:1323-1331.
22. Goldstein T, Anthony S J, Gbakima A, Bird B H, Bangura J, Tremeau-Bravard A, Belaganahalli M N, Wells H L, Dhanota J K, Liang E, Grodus M, Jangra R K, DeJesus V A, Lasso G, Smith B R, Jambai A, Kamara B O, Kamara S, Bangura W, Monagin C, Shapira S, Johnson C K, Saylors K, Rubin E M, Chandran K, Lipkin W I, Mazet J A K. 2018. The discovery of Bombali virus adds further support for bats as hosts of ebolaviruses. Nat Microbiol 3:1084-1089.
23. Heidecker B, Williams S H, Jain K, Oleynik A, Patriki D, Kottwitz J, Berg J, Garcia J A, Baltensperger N, Lovrinovic M, Baltensweiler A, Mishra N, Briese T, Hanson P J, Lauten A, Poller W, Leistner D M, Landmesser U, Enseleit F, McManus B, Luscher T F, Lipkin W I. 2020. Virome Sequencing in Patients With Myocarditis. Circ Heart Fail 13:e007103.
24. Kim K W, Horton J L, Pang C N I, Jain K, Leung P, Isaacs S R, Bull R A, Luciani F, Wilkins M R, Catteau J, Lipkin W I, Rawlinson W D, Briese T, Craig M E. 2019. Higher abundance of enterovirus A species in the gut of children with islet autoimmunity. Sci Rep 9:1749.
25. McGill F, Tokarz R, Thomson E C, Filipe A, Sameroff S, Jain K, Bhuva N, Ashraf S, Lipkin W I, Corless C, Pattabiraman C, Gibney B, Griffiths M J, Geretti A M, Michael B D, Beeching N J, McKee D, Hart I J, Mutton K, Jung A, Miller A, Solomon T. 2022. Viral capture sequencing detects unexpected viruses in the cerebrospinal fluid of adults with meningitis. J Infect 84:499-510.
26. Mishra N, Ng T F F, Marine R L, Jain K, Ng J, Thakkar R, Caciula A, Price A, Garcia J A, Burns J C, Thakur K T, Hetzler K L, Routh J A, Konopka-Anstadt J L, Nix W A, Tokarz R, Briese T, Oberste M S, Lipkin W I. 2019. Antibodies to Enteroviruses in Cerebrospinal Fluid of Patients with Acute Flaccid Myelitis. mBio 10.
27. Souza T M L, Vieira Y R, Delatorre E, Barbosa-Lima G, Luiz R L F, Vizzoni A, Jain K, Miranda M M, Bhuva N, Gogarten J F, Ng J, Thakkar R, Calheiros A S, Monteiro A P T, Bozza P T, Bozza F A, Tschoeke D A, Leomil L, Mendonca M C L, Rodrigues C, Torres M C, Filippis A M B, Nogueira R M R, Thompson F L, Lemos C, Durovni B, Cerbino-Neto J, Morel C M, Lipkin W I, Mishra N. 2019. Emergence of the East-Central-South-African genotype of Chikungunya virus in Brazil and the city of Rio de Janeiro may have occurred years before surveillance detection. Sci Rep 9:2760.
28. Tokarz R, Hyams J S, Mack D R, Boyle B, Griffiths A M, LeLeiko N S, Sauer C G, Shah S, Markowitz J, Baker S S, Rosh J, Baldassano R N, Kugathasan S, Walters T, Tagliafierro T, Sameroff S, Lee B, Che X, Oleynik A, Denson L A, Lipkin W I. 2019. Characterization of Stool Virome in Children Newly Diagnosed With Moderate to Severe Ulcerative Colitis. Inflamm Bowel Dis 25:1656-1662.
29. Williams S H, Cordey S, Bhuva N, Laubscher F, Hartley M A, Boillat-Blanco N, Mbarack Z, Samaka J, Mlaganile T, Jain K, d'Acremont V, Kaiser L, Lipkin W I. 2018. Investigation of the Plasma Virome from Cases of Unexplained Febrile Illness in Tanzania from 2013 to 2014: a Comparative Analysis between Unbiased and VirCapSeq-VERT High-Throughput Sequencing Approaches. mSphere 3.
30. Gogarten J F, Ulrich M, Bhuva N, Garcia J, Jain K, Lee B, Lohrich T, Oleynik A, Couacy-Hymann E, Fuh Neba T, Mishra N, Briese T, Calvignac-Spencer S, Lipkin W I, Leendertz F H. 2019. A Novel Orthohepadnavirus Identified in a Dead Maxwell's Duiker (Philantomba maxwellii) in Tai National Park, Cote d'Ivoire. Viruses 11.
31. Paulson J N, Williams B L, Hehnly C, Mishra N, Sinnar S A, Zhang L, Ssentongo P, Mbabazi-Kabachelor E, Wijetunge D S S, von Bredow B, Mulondo R, Kiwanuka J, Bajunirwe F, Bazira J, Bebell L M, Burgoine K, Couto-Rodriguez M, Ericson J E, Erickson T, Ferrari M, Gladstone M, Guo C, Haran M, Hornig M, Isaacs A M, Kaaya B N, Kangere S M, Kulkarni A V, Kumbakumba E, Li X, Limbrick D D, Jr., Magombe J, Morton S U, Mugamba J, Ng J, Olupot-Olupot P, Onen J, Peterson M R, Roy F, Sheldon K, Townsend R, Weeks A D, Whalen A J, Quackenbush J, Ssenyonga P, Galperin M Y, Almeida M, Atkins H, Warf B C, Lipkin W I, et al. 2020. Paenibacillus infection with frequent viral coinfection contributes to postinfectious hydrocephalus in Ugandan infants. Sci Transl Med 12.
32. Government US. 2015. Medicare, Medicaid, and CLIA Programs; Clinical Laboratory Improvement Amendments of 1988 Exemption of Permit-Holding Laboratories in the State of New York.
33. Mishra N, Ng J, Rakeman J L, Perry M J, Centurioni D A, Dean A B, Price A, Thakkar R, Angus A G, Williamson P, Delwart E, Carrington C, Sahadeo N, Che X, Briese T, Tokarz R, Lipkin W I. 2019. One-step pentaplex real-time polymerase chain reaction assay for detection of zika, dengue, chikungunya, West nile viruses and a human housekeeping gene. J Clin Virol 120:44-50.
34. PGM. 2020. ACCELERATED EMERGENCY USE AUTHORIZATION (EUA) SUMMARY The TRIPLEX CII-SARS-CoV-2 rRT-PCR TEST
35. Jiang L, Schlesinger F, Davis C A, Zhang Y, Li R, Salit M, Gingeras T R, Oliver B. 2011. Synthetic spike-in standards for RNA-seq experiments. Genome Res 21:1543-51.
36. Martin MJEj. 2011. Cutadapt removes adapter sequences from high-throughput sequencing reads. 17:10-12.
37. Schmieder R, Edwards R. 2011. Quality control and preprocessing of metagenomic datasets. Bioinformatics 27:863-4.
38. Langmead B, Salzberg S L. 2012. Fast gapped-read alignment with Bowtie 2. Nat Methods 9:357-9.
39. Rusnakova D, Sedlackova T, Radvak P, Bohmer M, Misenko P, Budis J, Bokorova S, Lipkova N, Forgacova-Jakubkova M, Sladecek T, Sitarcik J, Krampl W, Gaziova M, Kalinakova A, Staronova E, Ticha E, Vrablova T, Sevcikova L, Kotvasova B, Madarova L, Feikova S, Benova K, Reizigova L, Onderkova Z, Ondruskova D, Loderer D, Skerenova M, Dankova Z, Janikova K, Halasova E, Novakova E, Turna J, Szemes T. 2022. Systematic Genomic Surveillance of SARS-CoV-2 Virus on Illumina Sequencing Platforms in the Slovak Republic-One Year Experience. Viruses 14.
40. Wallace Z S, Davis J, Niewiadomska A M, Olson R D, Shukla M, Stevens R, Zhang Y, Zmasek C M, Scheuermann R H. 2022. Early detection of emerging SARS-CoV-2 variants of interest for experimental evaluation. Front Bioinform 2:1020189.
41. Hofman P, Bordone O, Chamorey E, Benzaquen J, Schiappa R, Lespinet-Fabre V, Lanteri E, Brest P, Mograbi B, Maniel C, Tanga V, Allegra M, Salah M, Fayada J, Boutros J, Leroy S, Heeke S, Hofman V, Marquette C H, Ilie M. 2021. Setting-Up a Rapid SARS-CoV-2 Genome Assessment by Next-Generation Sequencing in an Academic Hospital Center (LPCE, Louis Pasteur Hospital, Nice, France). Front Med (Lausanne) 8:730577.
42. Robles-Escajeda E, Mohl J E, Contreras L, Betancourt A P, Mancera B M, Kirken R A, Rodriguez G. 2023. Rapid Shift from SARS-CoV-2 Delta to Omicron Sub-Variants within a Dynamic Southern U.S. Borderplex. Viruses 15.
43. Babady N E, England M R, Jurcic Smith K L, He T, Wijetunge D S, Tang Y W, Chamberland R R, Menegus M, Swierkosz E M, Jerris R C, Greene W. 2018. Multicenter Evaluation of the ePlex Respiratory Pathogen Panel for the Detection of Viral and Bacterial Respiratory Tract Pathogens in Nasopharyngeal Swabs. J Clin Microbiol 56.
44. Chen J H K, Lam H Y, Yip C C Y, Wong S C Y, Chan J F W, Ma E S K, Cheng V C C, Tang B S F, Yuen K Y. 2016. Clinical Evaluation of the New High-Throughput Luminex NxTAG Respiratory Pathogen Panel Assay for Multiplex Respiratory Pathogen Detection. J Clin Microbiol 54:1820-1825.
45. Tang Y W, Gonsalves S, Sun J Y, Stiles J, Gilhuley K A, Mikhlina A, Dunbar S A, Babady N E, Zhang H. 2016. Clinical Evaluation of the Luminex NxTAG Respiratory Pathogen Panel. J Clin Microbiol 54:1912-1914.
46. Jullien S, Fitzgerald F, Keddie S, Baerenbold O, Bassat Q, Bradley J, Falconer J, Fink C, Keogh R, Hopkins H, Voice M. 2022. Diagnostic accuracy of multiplex respiratory pathogen panels for influenza or respiratory syncytial virus infections: systematic review and meta-analysis. BMC Infect Dis 22:785.
47. Masci A L, Menesale E B, Chen W C, Co C, Lu X, Bergelson S. 2019. Integration of Fluorescence Detection and Image-Based Automated Counting Increases Speed, Sensitivity, and Robustness of Plaque Assays. Mol Ther Methods Clin Dev 14:270-274.
48. Smither S J, Lear-Rooney C, Biggins J, Pettitt J, Lever M S, Olinger G G, Jr. 2013. Comparison of the plaque assay and 50% tissue culture infectious dose assay as methods for measuring filovirus infectivity. J Virol Methods 193:565-71.
49. Baylis S A, Wallace P, McCulloch E, Niesters H G M, Nubling C M. 2019. Standardization of Nucleic Acid Tests: the Approach of the World Health Organization. J Clin Microbiol 57.
50. Blauwkamp T A, Thair S, Rosen M J, Blair L, Lindner M S, Vilfan I D, Kawli T, Christians F C, Venkatasubrahmanyam S, Wall G D, Cheung A, Rogers Z N, Meshulam-Simon G, Huijse L, Balakrishnan S, Quinn J V, Hollemon D, Hong D K, Vaughn M L, Kertesz M, Bercovici S, Wilber J C, Yang S. 2019. Analytical and clinical validation of a microbial cell-free DNA sequencing test for infectious disease. Nat Microbiol 4:663-674.
51. Illumina. 2020. Illumina Respiratory Pathogen ID/AMR Panel.

What is claimed is:

1. A virome capture platform for the detection, identification, and/or characterization of vertebrate-infecting viruses in a sample,
the platform comprising a plurality of oligonucleotide probes, wherein, for each viral taxon included in the platform, the plurality comprises at least one probe partially or fully complementary to a portion of a predetermined coding sequence of said viral taxon,
wherein the predetermined coding sequences of the viral taxa, or fragments thereof, cluster at about 60-100% sequence identity,
wherein oligonucleotide probes partially or fully complementary to a portion of a predetermined coding sequence within a single viral taxon cluster at about 70-100% sequence identity,
wherein each oligonucleotide probe is about 50-300 nucleotides in length, preferably about 100-300 nucleotides in length,
wherein different oligonucleotide probes of the plurality which bind the same predetermined coding sequence are tiled across said predetermined coding sequence at intervals of about 50-500 nucleotides, and
wherein the plurality of oligonucleotide probes of the platform comprises 100,000 to 1,500,000 oligonucleotide probes, preferably less than about 1,000,000 oligonucleotide probes.

2. The platform of claim 1, wherein each oligonucleotide probe is about 100-300 nucleotides in length, preferably 100-150 nucleotides in length.

3. The platform of claim 1, wherein the average length of the plurality of oligonucleotide probes is about 125 nucleotides.

4. The platform of claim 1, wherein the melting temperature of each oligonucleotide probe is about 50-125° C.

5. The platform of claim 1, wherein different oligonucleotide probes which bind the same predetermined coding sequence are tiled across said predetermined coding sequence at about 60 nucleotide intervals.

6. The platform of claim 1, wherein oligonucleotide probes partially or fully complementary to a portion of a hepacivirus coding sequence cluster at about 92% sequence identity.

7. The platform of claim 1, wherein oligonucleotide probes partially or fully complementary to a portion of a primate lentivirus coding sequence cluster at about 85% sequence identity.

8. The platform of claim 1, wherein the predetermined coding sequences of the viral taxa, or fragments thereof, cluster at about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity.

9. The platform of claim 1, wherein each predetermined coding sequence comprises a portion which is partially or fully complementary to an oligonucleotide probe and the portion is about 50-300 nucleotides in length.

10. The platform of claim 1, wherein each oligonucleotide probe is at least 80% complementary, preferably at least 90% complementary, to a portion of a predetermined coding sequence.

11. The platform of claim 1, wherein each viral taxon included in the platform is listed in Table A.

12. The platform of claim 1, wherein the viruses infect at least one vertebrate organ or vertebrate organ system.

13. The platform of claim 12, wherein the at least one vertebrate organ is selected from the group consisting of skin, liver, brain, lungs, heart, kidney, stomach, intestines, colon, spleen, pancreas, and thyroid.

14. The platform of claim 1, wherein each oligonucleotide probe comprises a capture portion.

15. The platform of claim 14, wherein the capture portion is selected from the group consisting of biotin, digoxygenin, a ligand, a small organic molecule, a small inorganic molecule, an aptamer, an antigen, an antibody, and a substrate.

16. The platform of claim 1, wherein the sample is obtained from a human subject.

17. A method of screening a sample for vertebrate-infecting viruses, the method comprising:
exposing the sample, or nucleic acids isolated, amplified, and/or enriched from the sample, to the virome capture platform of claim 1 to form one or more hybridization products, wherein each hybridization product comprises a nucleic acid of the sample and an oligonucleotide probe of the platform;
capturing the one or more hybridization products; and
identifying the presence of one or more virus taxa in the sample based on the sequences of the one or more captured hybridization products;
thereby screening the sample for vertebrate-infecting viruses.

18. The method of claim 17, wherein nucleic acids in the sample are isolated and/or enriched prior to the exposing in step (a).

19. The method of claim 17, the method further comprising:
sequencing one or more detected hybridization products;
comparing the nucleotide sequence of the one or more hybridization products to nucleotide sequences of known viruses; and
identifying and/or characterizing one or more viruses in the sample based on sequence identity of the hybridization product to the nucleotide sequences of known viruses.

20. A kit comprising the virome capture platform of claim 1 and instructions for using the platform.
